(19) United States
(12) Patent Application Publication
(10) Pub. No.: US 2016/0114143 A1
(43) Pub. Date: Apr. 28, 2016

(54) ANTISEPTIC DELIVERY SYSTEM

(71) Applicant: MEDINOVEL INC., Calgary (CA)

(72) Inventors: Ryszard KOWALEWSKI, Calgary (CA); Alexandra KOWALEWSKI, Vancouver (CA); Marcin Rafal KOWALEWSKI, Calgary (CA)

(73) Assignee: MEDINOVEL INC., Calgary (CA)

(21) Appl. No.: 14/924,293

(22) Filed: Oct. 27, 2015

Related U.S. Application Data
(60) Provisional application No. 62/069,788, filed on Oct. 28, 2014.

(51) Int. Cl. A61F 13/40

(52) U.S. Cl. CPC A61M 35/006

(57) ABSTRACT

An agent delivery system may include an applicator including a hand piece having a hand-receiving cavity and an agent application material on a surface of the hand piece. The system may further include a fluid-containing reservoir, wherein the fluid-containing reservoir is configured to release fluid from the reservoir to allow the fluid to transfer onto the agent application material.

### CROSS-REFERENCE TO RELATED APPLICATION

This application claims the benefit of priority under 35 U.S.C. §119 to U.S. Provisional Patent Application No. 62/069,788, filed Oct. 28, 2014, the entirety of which is incorporated herein by reference.

### TECHNICAL FIELD

The present disclosure relates to the field of delivering antiseptic to a target that is to be disinfected, typically prior to skin incision during surgery and, in particular, to an antiseptic delivery system that may be used for surgical skin prep.

### BACKGROUND

Surgical site infection (SSI) can be a devastating complication of any surgical procedure. Patients who develop SSI have longer and costlier hospitalizations, are more likely to be readmitted, and have an increased morbidity and mortality. An SSI occurs when bacteria enter a surgical wound. Most commonly this happens during surgery, where bacteria can come from several sources, including the environment, the staff and the patient itself. Most SSIs are said to originate from the patient's own skin which is not properly disinfected prior to skin incision. Reducing the number of bacteria on the skin around the incision site reduces the risk of a patient developing an SSI. This can be achieved by applying in a very particular way antiseptics on the skin. This process combines mechanical friction and chemical killing. This practice is known as “skin prep” which involves the application of an antimicrobial solution to the patient's skin on and around the surgical site.

The conventional method of skin prep is to pour an antiseptic solution from the bottle onto a tray, dip a dry sponge or towelling into the antiseptic solution and apply the soaked sponge or towelling to the surface of skin using sterile technique. The antiseptic solution is applied on the skin site once or several times depending on the antiseptic being used and local (hospital) protocols. The sponge or towelling provides a physical means of spreading the antiseptic solution evenly, and rubbing the skin can help remove dead skin cells and contaminated skin debris.

Multi-step surgical prep kits are typically used. These standard kits generally include a plastic tray, drape towel, pair of sterile gloves, and a variety of absorbents such as sponges, tissues, and swabs. The antiseptic solution is either included in the kit or separately provided. The multi-component nature of these prep kits make them bulky and cumbersome to both store and handle. Moreover, these kits tend to be awkward and time-consuming to implement by a single person, and present opportunities for contamination to be introduced in the multi-step operation of dispensing the antiseptic preparation, donning gloves and preparing the applicator while maintaining sterile conditions.

Surgical prep kits that comprise pre-packaged and pre-soaked sponges or swabs are also available. The pre-soaked sponges are attached to a blade or held with forceps, or can be provided with an attached handle. These pre-soaked sponges are conveniently provided with enough antiseptic preparation to saturate the sponge so as to reduce the number of steps involved in the conventional method of pre-operative skin prep. These kits, however, are messy and offer little control over inadvertent dripping of the solution into areas where it is undesired. As well, the pre-soaked sponges or swabs cover a relatively small surface area and are difficult to use for accessing large or difficult areas of the body, such as the leg, arm, foot, and hand.

Single use, self-contained applicator systems have also been developed to address some of the noted shortcomings of conventional systems. These applicator systems generally contain a single-use dose of the antiseptic preparation in a container attached to a sponge pad to apply a layer of the antiseptic preparation to the skin.

U.S. Pat. No. 6,488,665 describes antimicrobial skin-preparation delivery systems that include a sealed, flexible container and an antimicrobial gel formulation dispenser connected to the container. The gel formulation dispenser includes a container connector connected to the flexible container and a gel applicator having a sealed piercing member which is slidably engaged with the container connector. An applicator pad is porous and also has enlarged holes for passage of the gel formulation. The gel formulation dispenser pierces a seal in the container and delivers the antimicrobial alcohol gel formulation from the container to the surgical site. Flow rate of the gel formulation is largely controlled by the amount of external pressure of squeezing a user applies to the flexible container.

U.S. Pat. No. 8,105,306 similarly describes a skin antiseptic composition dispenser that includes a container and an applicator with a hydrophilic foam.

While such systems are designed to improve the efficiency of conventional multi-step surgical prep systems, these self-contained applicator systems offer limited versatility for use in difficult to access areas of the body, for example, the foot, hand, groin, etc. As well, these systems can be complicated in design and, therefore, difficult and costly to manufacture. Accordingly, skin prep delivery systems that address these shortcomings are highly desirable and will improve the efficiency and reduce waste of conventional surgical prep systems.

This background information is provided for the purpose of making known information believed by the applicant to be of possible relevance to the present disclosure. No admission is necessarily intended, nor should be construed, that any of the preceding information constitutes prior art against the present disclosure.

### SUMMARY

Disclosed herein are exemplary embodiments pertaining to an antiseptic delivery system, sometimes referred to herein as a surgical skin prep system. An exemplary embodiment of the present disclosure relates to a self-contained antiseptic delivery system, comprising an applicator for distributing an antiseptic composition onto a target body surface, the applicator comprising a hand article or hand piece having an inside and outside surface wherein the inside surface receives a user's hand at a hand-receiving end, and the hand article comprising an absorbent material that may be saturated with the antiseptic composition. A protective sheath encasing the hand piece, the protective sheath being releasable to expose the applicator when in use may be utilized. The protective sheath maintains the hand piece in an aseptic enclosure. According to further embodiments an external sheath within which the encased applicator is housed; and a plurality of restrictors or connectors attaching the external sheath to the protective sheath at opposing surfaces of the hand article, wherein the plurality of restrictors or connectors maintain the applicator in a stored position when not in use, and wherein the plurality of restrictors or connectors permit the applicator to be released in an aseptic manner, into a deployed position when in use.

In accordance with another aspect of the disclosure, there is provided a kit for disinfecting an incision site in preparation for surgery, the kit comprising the self-contained antiseptic delivery system according to embodiments described in the present disclosure.

In accordance with a further aspect of the disclosure, there is provided a method for disinfecting an incision site in preparation for surgery comprising the self-contained antiseptic delivery system according to embodiments described in the present disclosure.

### BRIEF DESCRIPTION OF THE DRAWINGS

These and other features of the examples provided herein will become more apparent in the following detailed description in which reference is made to the appended drawings.

FIGS. 1 through 12 are views of an exemplary and first illustrated embodiment of an antiseptic delivery system, according to the present disclosure. Specifically,

FIG. 1 is a plan view of the mitt portion of the antiseptic delivery system, without illustrating the various coverings;

FIG. 2 is a plan view of the mitt shown in FIG. 1 and showing additional components;

FIG. 3 is a plan view of the mitt of FIG. 1 and showing additional components;

FIG. 4 is a plan view of the mitt of FIG. 1 and showing additional components;

FIG. 5 is a plan view of the mitt of FIG. 1 and showing additional components;

FIG. 6 is a plan view of the mitt of FIG. 1 and showing additional components;

FIG. 7 is a plan view of the mitt shown in FIG. 6, illustrating a first step in the deployment from the storage position toward the use position;

FIG. 8 is a plan view of the mitt shown in FIG. 7 and is a next sequential step in the deployment, showing the mitt in the fully deployed, use position;

FIG. 9 is a cross sectional view of the mitt illustrated in FIG. 6, taken along the line 9-9 of FIG. 6;

FIG. 10 is a cross sectional view of the mitt illustrated in FIG. 7, taken along the line 10-10 of FIG. 7 and showing a first step in the deployment;

FIG. 11 is a cross sectional view of the mitt illustrated in FIG. 8, taken along the line 11-11 of FIG. 8 and showing the next sequential step in the deployment;

FIG. 12 is a detail view of the close up circle in FIG. 9;

FIGS. 13 through 24 are views of an exemplary and second illustrated embodiment of an antiseptic delivery system, according to the present disclosure. Specifically,

FIG. 13 is a plan view of the mitt portion of the second illustrated embodiment of an antiseptic delivery system, with the various coverings removed to illustrate the mitt;

FIG. 14 is a plan view of the mitt shown in FIG. 13 and showing additional components;

FIG. 15 is a plan view of the mitt of FIG. 13 and showing additional components;

FIG. 16 is a plan view of the mitt of FIG. 13 and showing additional components;

FIG. 17 is a plan view of the mitt of FIG. 13 and showing additional components;

FIG. 18 is a plan view of the mitt of FIG. 13 and showing additional components;

FIG. 19 is a plan view of the mitt shown in FIG. 18, illustrating a first step in the deployment from the storage position toward the use position;

FIG. 20 is a plan view of the mitt shown in FIG. 19 and is a next sequential step in the deployment, showing the mitt in the fully deployed, use position;

FIG. 21 is a cross sectional view of the mitt illustrated in FIG. 18, taken along the line 21-21 of FIG. 18;

FIG. 22 is a cross sectional view of the mitt illustrated in FIG. 19, taken along the line 22-22 of FIG. 19 and showing a first step in the deployment;

FIG. 23 is a cross sectional view of the mitt illustrated in FIG. 20, taken along the line 23-23 of FIG. 20 and showing the next sequential step in the deployment;

FIG. 24 is a detail view of the close up circle in FIG. 21;

FIGS. 25 through 35 are views of an exemplary and third illustrated embodiment of an antiseptic delivery system, according to the present disclosure. Specifically,

FIG. 25 is a plan view of the mitt portion of the third illustrated embodiment of an antiseptic delivery system, with the various coverings removed to illustrate the mitt;

FIG. 26 is a plan view of the mitt shown in FIG. 25 and showing additional components;

FIG. 27 is a plan view of the mitt of FIG. 25 and showing additional components;

FIG. 28 is a plan view of the mitt of FIG. 25 and showing additional components;

FIG. 29 is a plan view of the mitt of FIG. 25 and showing additional components;

FIG. 30 is a plan view of the mitt shown in FIG. 29, illustrating a first step in the deployment from the storage position toward the use position;

FIG. 31 is a plan view of the mitt shown in FIG. 30 and is a next sequential step in the deployment, showing the mitt in the fully deployed, use position;

FIG. 32 is a cross sectional view of the mitt illustrated in FIG. 29, taken along the line 32-32 of FIG. 29;

FIG. 33 is a cross sectional view of the mitt illustrated in FIG. 30, taken along the line 33-33 of FIG. 30 and showing a first step in the deployment;

FIG. 34 is a cross sectional view of the mitt illustrated in FIG. 32, taken along the line 34-34 of FIG. 32 and showing the next sequential step in the deployment;

FIG. 35 is a detail view of the close up circle in FIG. 32;

FIGS. 36 through 46 are views of an exemplary and fourth illustrated embodiment of an antiseptic delivery system, according to the present disclosure. Specifically,

FIG. 36 is a plan view of the mitt portion of the fourth illustrated embodiment of an antiseptic delivery system, with the various coverings removed to illustrate the mitt;

FIG. 37 is a plan view of the mitt shown in FIG. 36 and showing additional components;

FIG. 38 is a plan view of the mitt of FIG. 36 and showing additional components;

FIG. 39 is a plan view of the mitt of FIG. 36 and showing additional components;

FIG. 40 is a plan view of the mitt of FIG. 36 and showing additional components;

FIG. 41 is a plan view of the mitt shown in FIG. 40, illustrating a first step in the deployment from the storage position toward the use position;

FIG. 42 is a plan view of the mitt shown in FIG. 40 with the mitt fully deployed in the use position;

FIG. 43 is a cross sectional view of the mitt shown in FIG. 40, taken along the line 43-43 of FIG. 40;

FIG. 44 is a cross sectional view of the mitt shown in FIG. 41, taken along the line 44-44 of FIG. 41;

FIG. 45 is a cross sectional view of the fully deployed mitt shown in FIG. 42, taken along the line 45-45 of FIG. 42;

FIG. 46 is a detail view of the close up circle in FIG. 43;

FIGS. 47 through 47G are views of a fifth illustrated embodiment of an antiseptic delivery system according to the present disclosure. Specifically,

FIG. 47 is a plan view of an exemplary and fifth illustrated embodiment of an antiseptic delivery system according to the present disclosure that incorporates a sealed and rupturable ampule containing a reservoir of disinfecting solution or other solution to be applied to the delivery system;

FIG. 47A is a cross sectional view of an embodiment shown in FIG. 47;

FIG. 47B is a cross sectional view of an exemplary ampule for use with the embodiment of FIG. 47, showing a fluid-containing reservoir of the ampule in a sealed condition;

FIG. 47C is a plan view of one embodiment of the ampule of FIG. 47;

FIG. 47D is a cross section of the ampule shown in FIG. 47B with the fluid-containing reservoir of the ampule in a ruptured condition;

FIG. 47E is a plan view of the ampule shown in FIG. 47C with the fluid-containing reservoir of the ampule in a ruptured condition;

FIG. 47F is a schematic cross sectional view of the ampule shown in FIG. 47D and illustrating the controlled dispersion of solution from the ruptured fluid-containing reservoir;

FIG. 47G is a schematic plan view of the ampule shown in FIG. 47F;

FIG. 47H is a plan view of an alternative embodiment of the ampule shown in FIG. 47C;

FIG. 47I is a plan view of yet another alternative embodiment of the ampule shown in FIG. 47C;

FIG. 47J is a cross sectional view of another embodiment of an ampule similar to the view of FIG. 47D;

FIG. 47K is a cross sectional view of an ampule illustrating a perforated layer that includes a porous material;

FIG. 47L is a cross sectional view of an ampule in which more than one perforated or porous layer is positioned between the fluid-containing reservoir and the absorbent material;

FIG. 47M is a cross sectional view of an ampule in which a perforated later encloses the fluid-containing reservoir;

FIG. 47N illustrates a fluid-containing reservoir positioned between an inner lining and an external surface of the absorbent material;

FIG. 47O illustrates an applicator in an enclosure;

FIG. 47P illustrates another example of an applicator in an enclosure;

FIG. 47Q illustrates the opening of a reservoir according to a first example;

FIG. 47R illustrates the opening of a reservoir according to a second example;

FIG. 47S illustrates the opening of a reservoir according to a third example;

FIG. 47T illustrates the opening of a reservoir according to a fourth example;

FIGS. 48 through 50 are views of an exemplary and sixth illustrated embodiment of an antiseptic delivery system, according to the present disclosure. Specifically,

FIG. 48 is a cross sectional view of a sixth illustrated embodiment of an antiseptic delivery system according to the present disclosure;

FIG. 49 is a cross sectional view of the embodiment of FIG. 48, showing the system in a first stage of deployment;

FIG. 50 is a cross sectional view of the embodiment of FIG. 48, showing the system in a fully deployed condition;

FIG. 51 is a cross sectional view of a seventh illustrated embodiment of an antiseptic delivery system; and

FIGS. 52 through 60 are a series of views of an antiseptic delivery system according to the present disclosure. Specifically

FIG. 52 is a plan view of an embodiment of an antiseptic delivery system that incorporates an applicator tip sterilizing system.

FIG. 53 is a plan view of the embodiment of FIG. 52 showing a next sequential step in the formation of the applicator tip sterilizing system.

FIG. 54 is a plan view of the embodiment of FIG. 53 showing a next sequential step in the formation of the applicator tip sterilizing system.

FIG. 55 is a plan view of the embodiment of FIG. 54 showing a next sequential step in the formation of the applicator tip sterilizing system.

FIG. 56 is a perspective view of the embodiment of FIG. 55 showing a next sequential step in the formation of the applicator tip sterilizing system.

FIG. 57 is a schematic cross sectional view taken along the line 57-57 of FIG. 54.

FIG. 58 is a schematic cross sectional view of the embodiment of FIG. 57, illustrating additional components of the embodiment.

FIG. 59 is a schematic cross sectional view of an alternative embodiment of the system shown in FIG. 57.

FIG. 60 is a schematic cross sectional view of the embodiment of FIG. 59, illustrating additional components of the embodiment.

FIG. 61 is a cross sectional view of an eighth illustrated embodiment of an antiseptic delivery system.

FIG. 62 is a plan view of a mitt similar to the mitt shown in FIG. 7, showing the mitt in the fully deployed position, and illustrating a colour coding system that may be utilized to illustrate to users the sterility or non-sterility of various portions of the device.

### DETAILED DESCRIPTION OF ILLUSTRATED EMBODIMENTS

### Definitions

Unless defined otherwise, all technical and scientific terms used herein have the same meaning as commonly understood by one of ordinary skill in the art to which this disclosure belongs.

As used herein, the terms “hand article” and “hand piece” refers to a covering for the hand and includes, for example, a glove or mitten. The term “glove” refers to a covering for the hand having separate sections for each finger. The term “mitten” or “mitt” refers to a covering for the hand having an enclosure that leaves the fingers unseparated and that may include space for the thumb in the main enclosure or may provide space for the thumb in a separate enclosure for the thumb or may not include a thumb enclosure at all.

As used herein, the term “disposable” describes articles that are not intended to be restored or reused and which are intended to be discarded after a single use.

As used herein, the term “about” refers to an approximately +/−10% variation from a given value. It is to be understood that such a variation is always included in any given value provided herein, whether or not it is specifically referred to.

Relative directional terms are used at times herein to describe to various structures. Generally, the term “inner” will refer to the relative position of the referenced structure that lies toward the user's hand when the mitt is being worn on the hand of a user. Other relative directional terms correspond to this naming convention: outer or the direction outwardly refers to the direction opposite inner or inwardly. The word “hermetically” is used at times herein to refer to a seal between various components of the disclosed devices and to the sterile condition that is established by the various seals. It should be understood that as used herein, hermetically or hermetical refers to a seal that defines a barrier between a medically sterile zone and a medically unsterile zone, or such a barrier between sterile zones. It will be understood by those of skill in the art that the word “sterility” is a relative term that, depending upon the context in which it is used, may have different meanings and connotations. As used herein, the word refers to an aseptic or uncontaminated condition. In that sense, “sterile” does not always refer to a condition where absolute sterility exists; an aseptic condition in a surgical sense typically means the exclusion of harmful or unwanted microorganisms.

The system and method according to the present disclosure provide for efficient and effective antiseptic delivery and surgical skin prep. The system, according to embodiments of the disclosure, comprises a sterile applicator that is pre-loaded with an antiseptic preparation. In this way, the system of the present disclosure is self-contained, thereby eliminating the need for multiple pre-packaged components. As a result, the number of steps required for performing pre-operative skin prep can be minimized, and often can be performed by a single person. The self-contained design of the system further reduces waste and simplifies disposal of the system after use.

The design of the system, according to the present disclosure, is self-contained, compact, light weight, and flexible thereby facilitating storage and transportability of the system. The system of the present disclosure is, therefore, amenable for use in extreme environments where economy of space and the need for transportability are necessary.

The system of the present disclosure comprises an applicator that is a hand article. In this way, a user is able to directly control the application of the antiseptic preparation irrespective of the size and intricacies of the target surgical site. For example, areas of the body that have multiple creases and parts, such as the hand or foot, can be effectively and specifically accessed for pre-operative skin prep without inadvertent dripping or pooling of the antiseptic preparation in non-target areas.

### System Construction

### Applicator

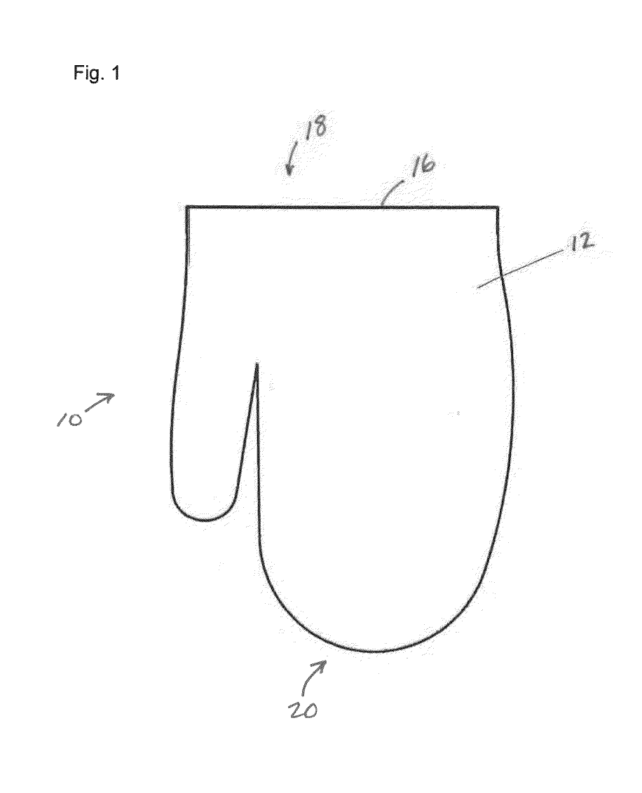

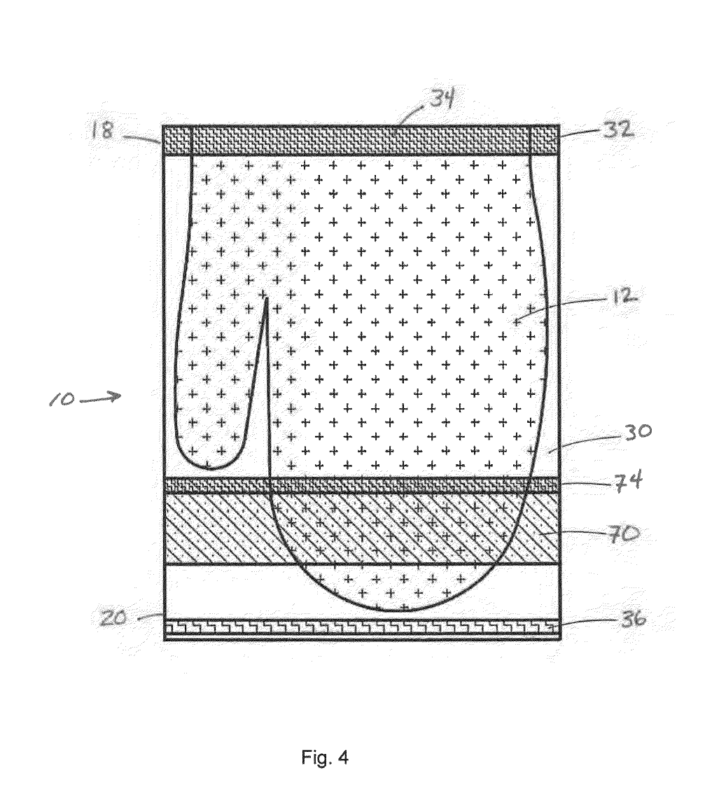

Referring now to the drawings, in which like reference numerals are used to identify identical or substantially similar parts throughout the several views, FIGS. 1 through 12 illustrate a first embodiment of the present disclosure. The antiseptic delivery system, which is referred to generally with reference number 10, comprises an applicator 12 that is provided pre-loaded with an antiseptic preparation. The applicator 12 is provided in the form of a hand article, also referred to at times as a hand piece, that has an internal cavity 14, which is accessible through an opening 16 at the hand-receiving end 18 and extends inwardly to the opposite end 20, which is closed and sealed to define a sterile barrier between the internal cavity 14 and the applicator 12 externally, or outwardly of the internal cavity. In use, the user inserts a hand into the hollow interior of the hand article, applicator 12, that is defined by internal cavity 14, through the opening 16 provided at the hand-receiving end 18. It will be appreciated, as detailed below, that when delivery system 10 is in use, the internal cavity 14 is an unsterile zone since the user's hand is inserted into the cavity, while the applicator 12 externally of the cavity 14 is a sterile zone.

As shown in the figures, the hand piece defined by applicator 12 may take the form of a mitt. The generic design of a mitt allows the applicator 12 to be used without regard to the handedness of the user, in other words, for either the user's left hand or right hand, and easily accommodates a relatively wide range of hand sizes. Although the system is described in reference to a mitt design, it is contemplated that a variety of hand shapes can be used. For example, a glove design, a fingerless hand covering, or a finger covering can be used.

With reference to the cross sectional view of FIG. 11, applicator 12 is constructed of a flexible liner 22 that defines the bounds of the internal cavity 14 that receives the user's hand, and an absorbent material 24 that covers the external surface of the liner 22 and which can be saturated with an antiseptic solution. The internal liner 22 corresponds in shape with the applicator 12 and defines the internal cavity 14. In this way, the liner 22 defines a barrier between the user's hand (which is inserted into cavity 14) and the absorbent material 24, thereby preventing contact between the user's hand that is inserted into the cavity 14 through opening 16 and the absorbent material 12 and maintaining sterility of the applicator 12 externally of liner 22. The absorbent material 24 may be capable of absorbing and retaining a desired volume of antiseptic preparation for application onto a target body part when performing pre-operative skin prep, or any target surface to which antiseptic is to be applied. Such absorbent materials are known in the art and include, for example, absorbent foam materials such as hydrophilic polyurethane foams, wipes and tissues. The absorbent material 24 may be any material that is configured to deliver an agent to a surface and may therefore be referred to as an agent application material. The agent applied by the agent application material may be in any suitable form (e.g., fluid, solid, gel, powder, etc.). The absorbent materials will be compatible with the antiseptic preparations and/or fluids and/or agents used for the particular application, and if needed will be able to withstand sterilization techniques such as pressurized steam, heat, radiation, and chemical techniques. The absorbent material 24 may be applied to the outer surface of liner 22 in any conventional manner such as laminating or bonding with adhesives.

The dimensions and thickness of the absorbent material 24 of applicator 12 can be adjusted to control the volume of the antiseptic preparation that is intended to be retained by the absorbent material 24. For use in preparation for a small surgical procedure, for example, the applicator 12 may be designed to retain a pre-loaded volume of the antiseptic preparation to generally be able to cover an area of, e.g., 1,000-2,000 square centimeters or more. In such embodiments, the applicator 12 can be pre-loaded with a volume of about 5-15 milliliters (ml) of an antiseptic preparation. For larger surgical procedures, the applicator 12 can be pre-loaded to provide an amount of antiseptic preparation to cover, for example, at least about 5,000 square centimeters. To cover a larger area, embodiments of the present disclosure comprise an applicator 12 that can be pre-loaded with a larger volume of antiseptic, for example, between at least about 20 ml to 30 ml of an antiseptic preparation. In this way, the system of the present disclosure can be tailored to the area of the body part to be disinfected, thereby, minimizing waste and cost. The foregoing volumes and dimensions are of course meant to be exemplary and do not limit the disclosure in any way.

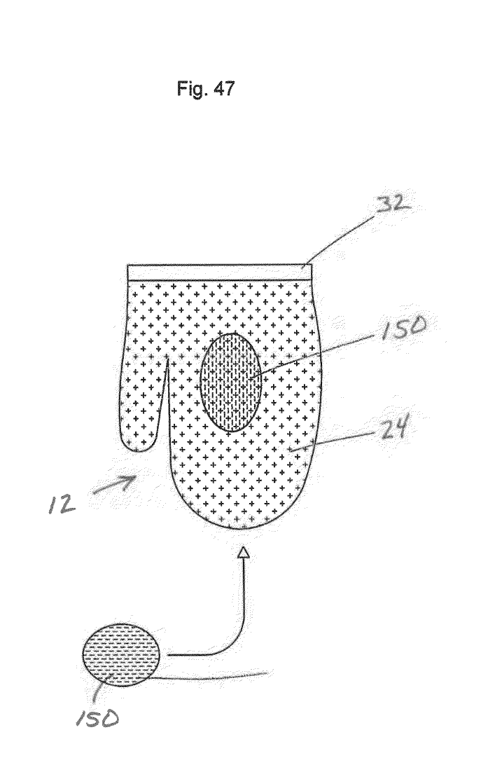

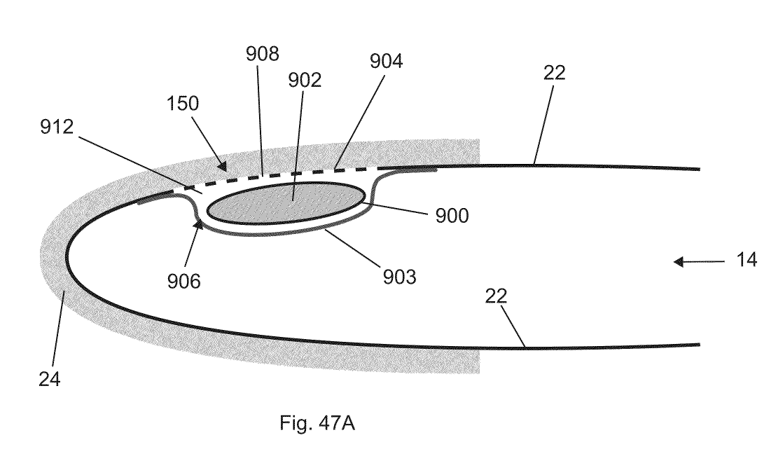

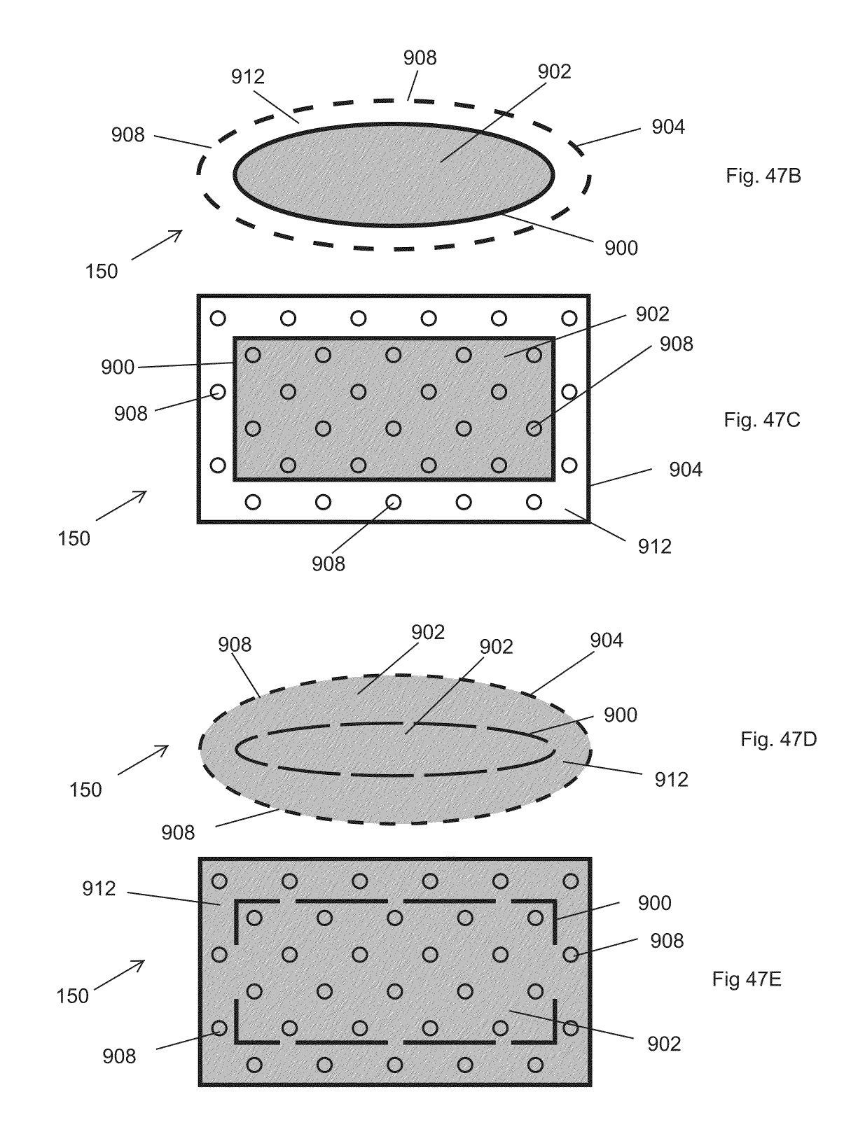

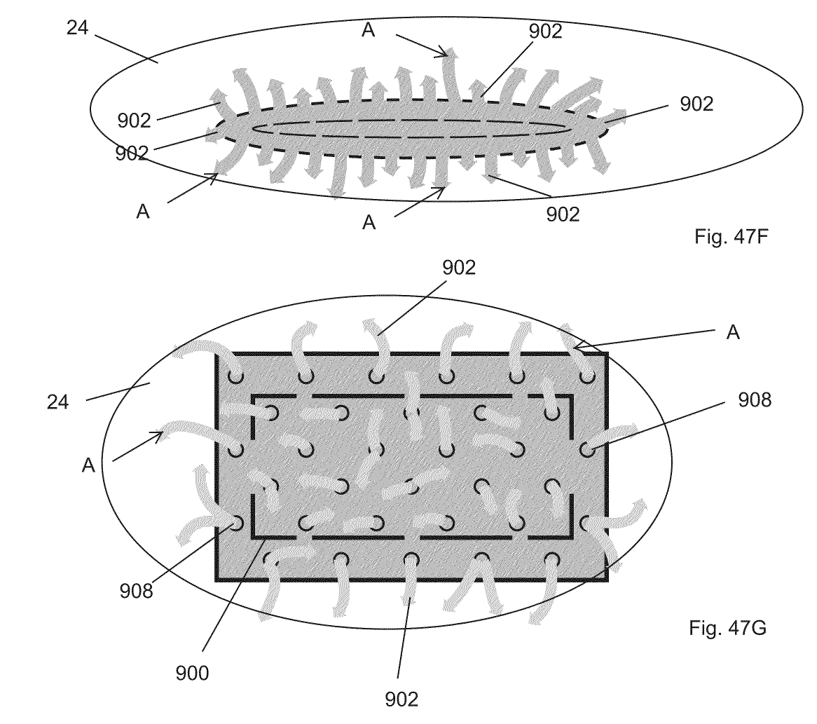

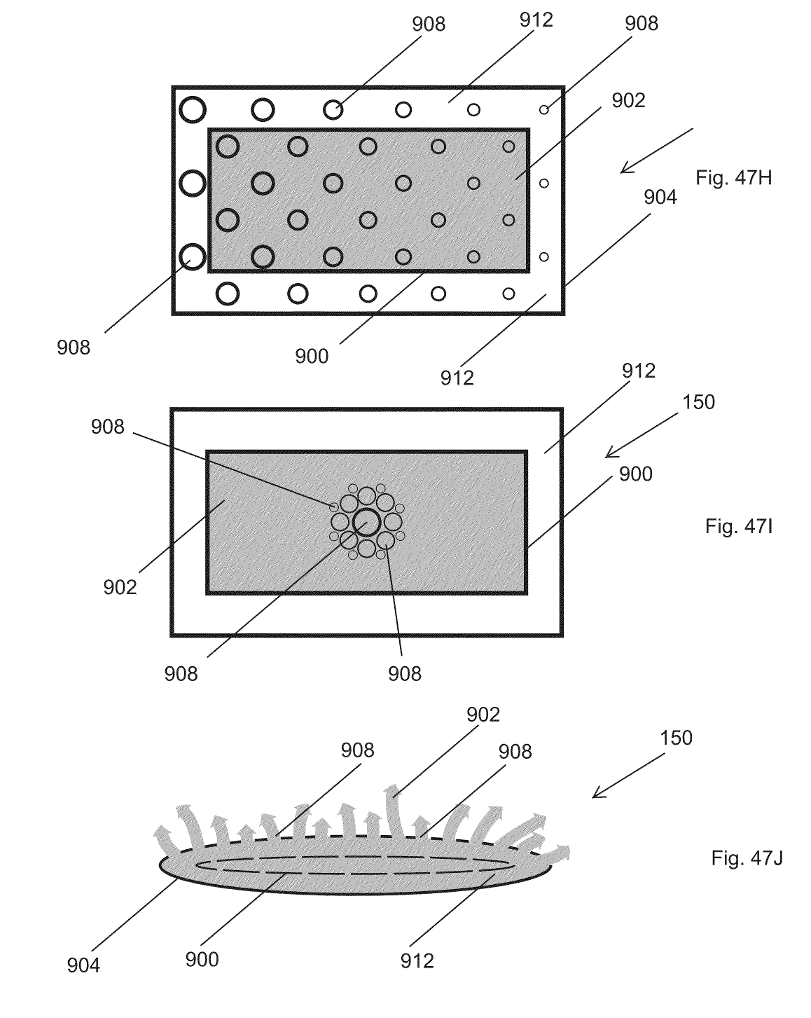

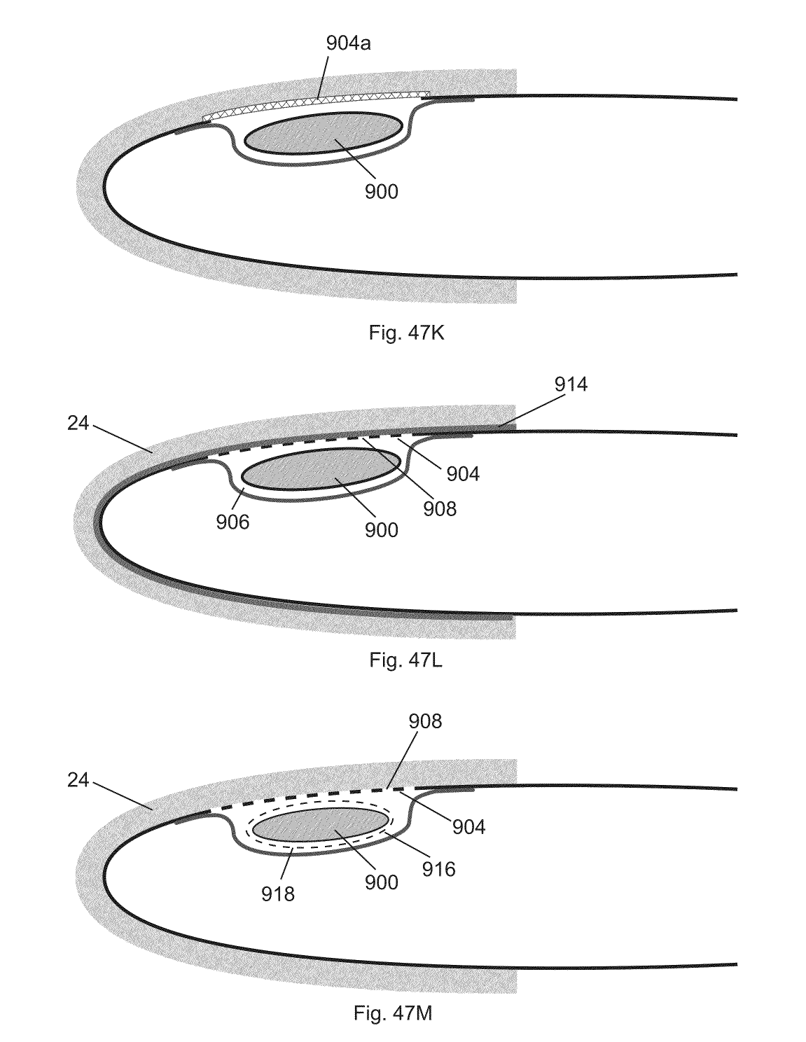

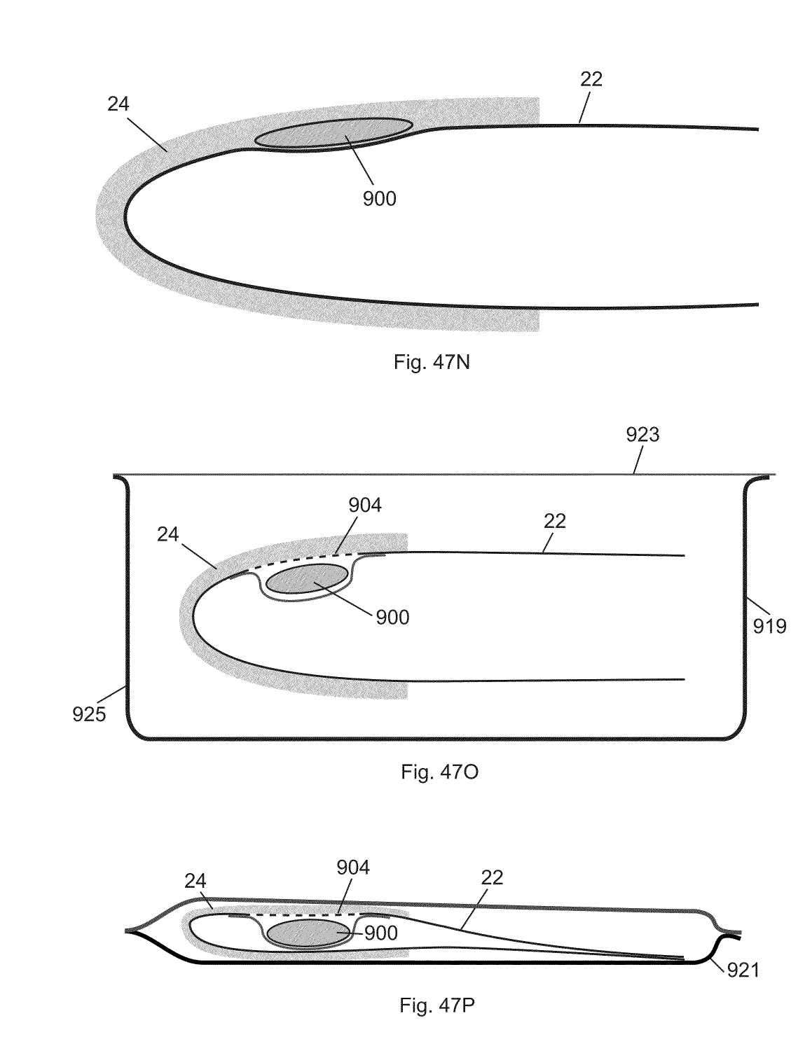

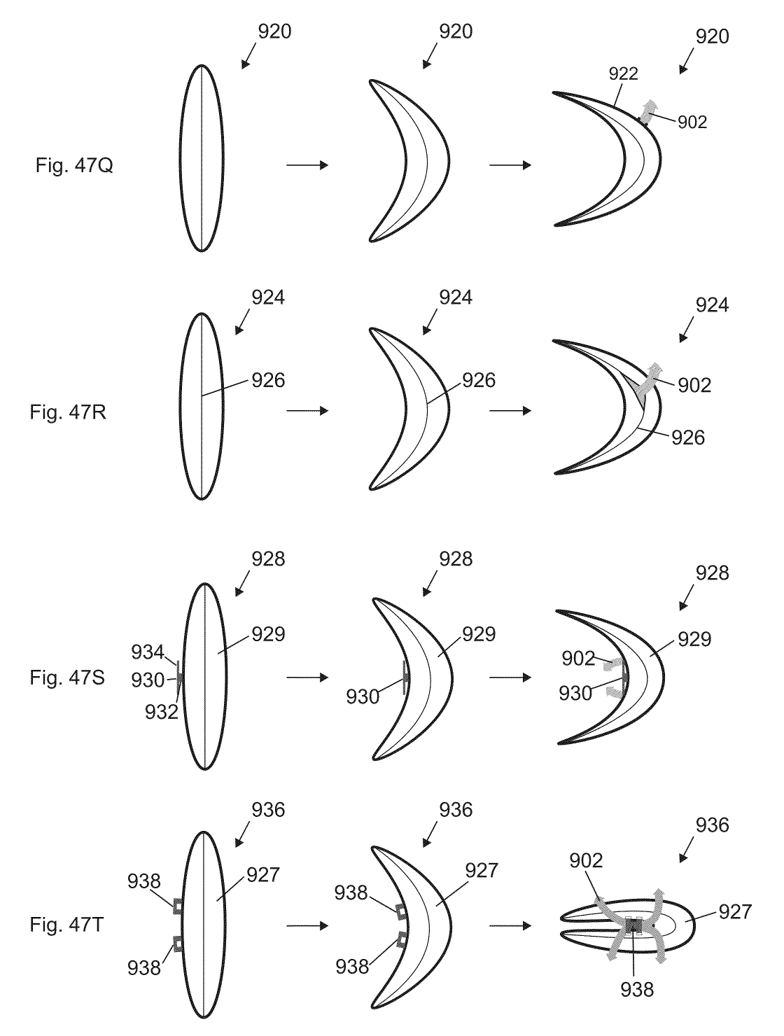

The applicator 12 is preloaded with an antiseptic preparation (or some other fluid, agent or medicament, depending upon the intended use of the applicator) thereby eliminating the time-consuming and awkward step of separately dispensing and applying the antiseptic preparation required by conventional antiseptic systems. In certain embodiments, the absorbent material 24 is pre-saturated with a desired volume of an antiseptic preparation. In other embodiments such as that shown in FIG. 47, a desired volume of an antiseptic preparation is contained in a separately sealed container or ampule 150 that is provided with the delivery system 10, to be released and absorbed into the absorbent material 24 of applicator 12 when ready to use. In the embodiment illustrated in FIG. 47, the antiseptic preparation is retained in ampule 150 between the applicator 12 and the protective sheath (discussed in further detail below) that protects the applicator 12 during storage to maintain a sterile environment. In such embodiments, the ampule 150 may be a squeezable capsule or a container, for example, which ruptures when squeezed to thereby release the antiseptic preparation retained in the container and onto the absorbent material 24.

The applicator 12 used in connection with the system of the present disclosure may be filled with various fluids or agents, such as antimicrobial agents, antiseptics, medicaments, cleansing preparation, chemical compositions, or the like. For example some suitable antimicrobial agents include, but are not limited to, iodine, an iodine complex, chlorhexidine, chlorhexidine salts, alcohol, or combinations thereof. Preferred iodine complexes may include iodophors, e.g., povidone-iodine USP. Preferred chlorhexidine salts may include, e.g., chlorhexidine digluconate, chlorhexidine diacetate. Other suitable antimicrobial agents may include C2-C5 lower alkyl alcohols (including, e.g., ethyl alcohol, 1-propanol, and 2-propanol), parachlorometaxylenol (PCMX), triclosan, hexachlorophene, fatty acid monoesters of glycerin and propylene glycol such as glycerol monolaurate, glycerol monocaprylate, glycerol monocaprate, propylene glycol monolaurate, propylene glycol monocaprylate, propylene glycol monocaprate, phenols, surfactants, and polymers that include a (C12-C22) hydrophobe and a quaternary ammonium group, polyquaternary amines such as polyhexamethylene biguanide, quaternary ammonium silanes, silver, silver salts (such as silver chloride), silver oxide and silver sulfadiazine, methyl, ethyl, propyl and butyl parabens, octenidene, peroxides (e.g., hydrogen peroxide and benzoyl peroxide), and the like, as well as combinations thereof.

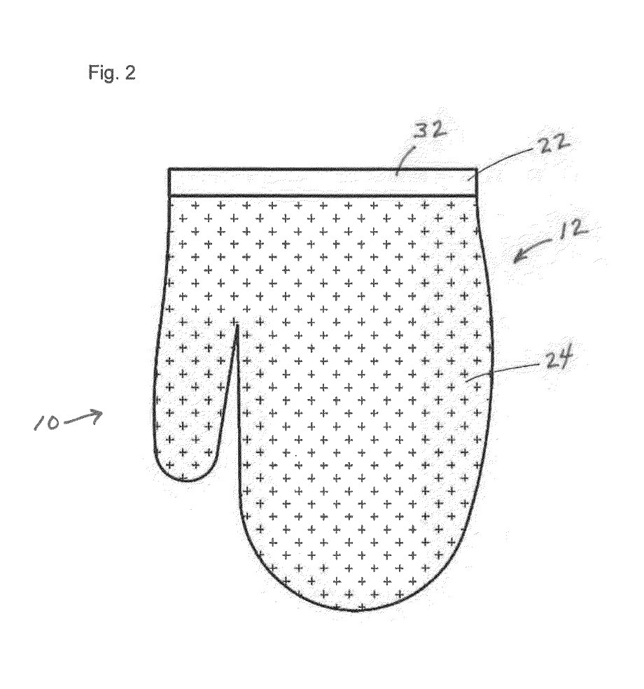

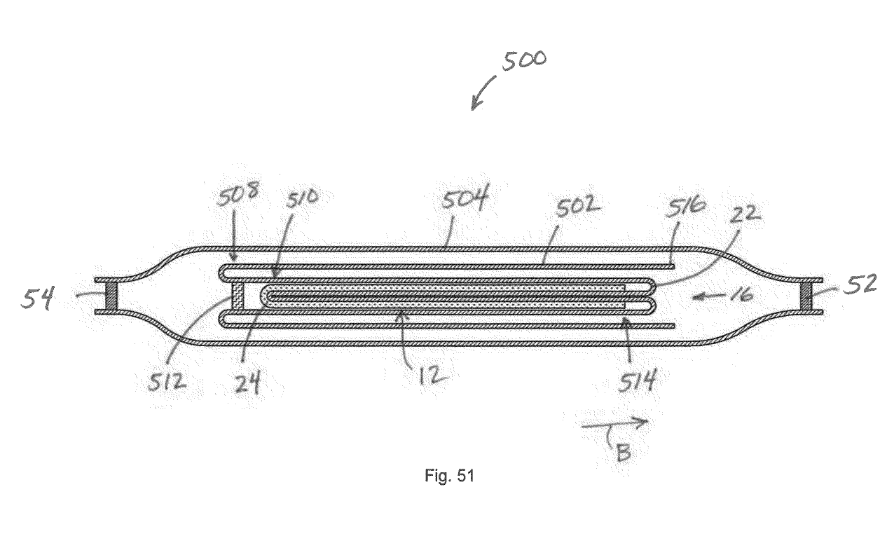

As shown in FIG. 2, the liner 22 extends slightly beyond the edge of the absorbent material 24 at the hand-receiving end 18 of the applicator 12 to provide an attachment flap 32 to which a protective sheath is attached as detailed below. And as shown in FIG. 51 and as detailed below, the liner 22 may be extended in length to act as a protective sheath.

### Aseptic Protective Sheath

Sterility of the applicator 12, and pre-loaded antiseptic preparation that is applied to absorbent material 24, must be maintained until the time of use. Accordingly, the applicator 12 must be stored in an aseptic environment and must also be readily usable under aseptic conditions. According to embodiments of the present disclosure, the self-contained skin prep system comprises a hermetically sealed protective sheath that encases the applicator 12 and maintains aseptic conditions during storage. The sterility of the applicator is insured when the applicator is retained within the protective sheath described below based on two elements. First, the protective sheath completely encases the applicator 12 in a sealed, sterile field. Second, the applicator is saturated with antiseptic, which defines a self-sterilizing element within the sterile enclosure defined by the protective sheath. The applicator 12 is exposed for use upon its release from the protective sheath.

The protective sheath defines an aseptic enclosure for applicator 12 and is referred to generally with reference no. 30. Protective sheath 30 will first be described with respect to the embodiment of the delivery system 10 illustrated in FIGS. 1 through 12.

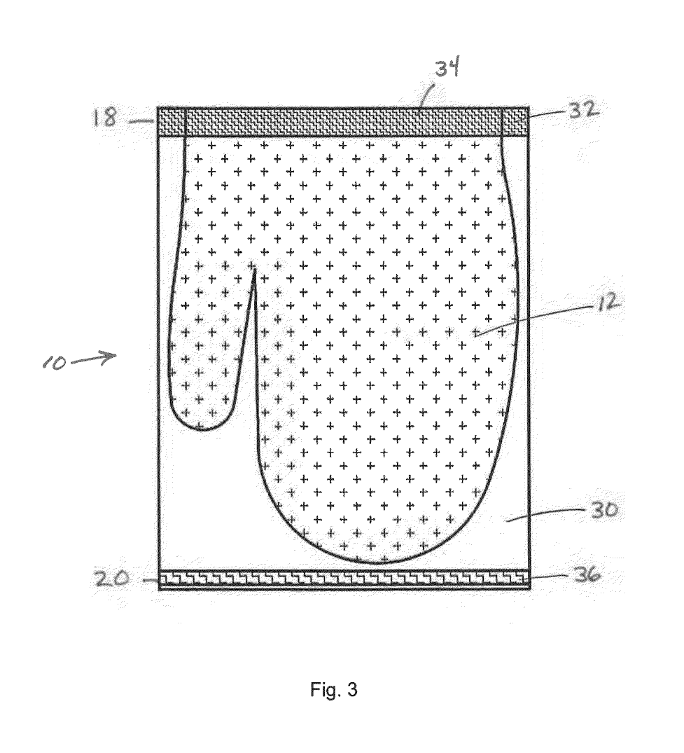

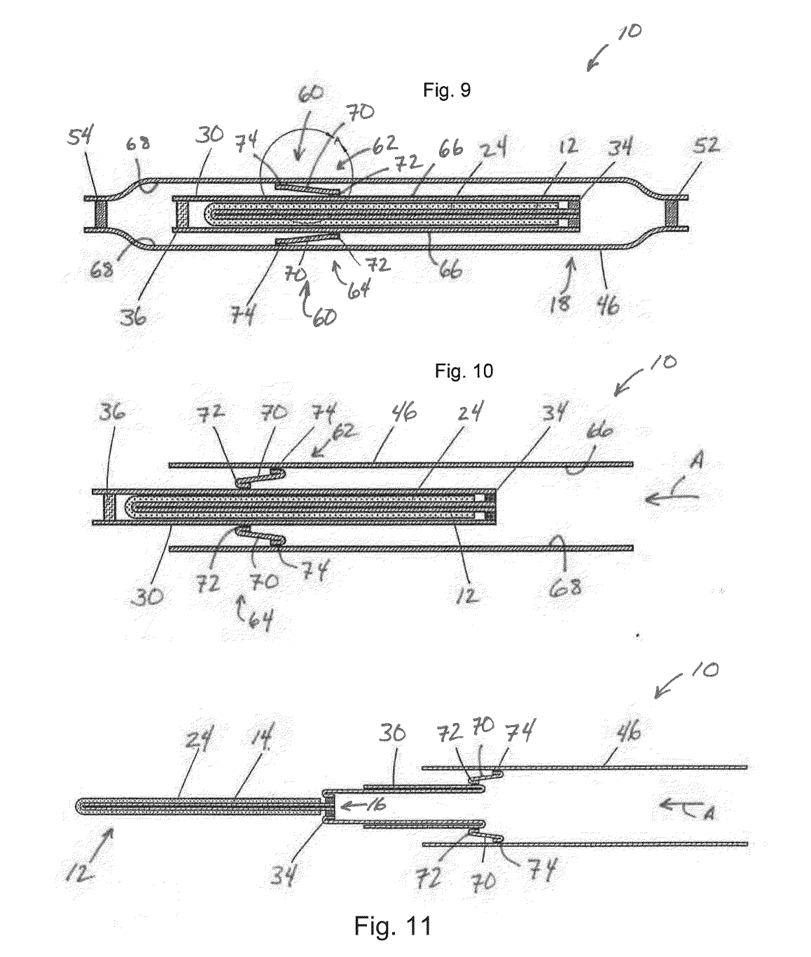

With reference now to FIG. 3, protective sheath 30 entirely surrounds and encloses the applicator 12 to define an aseptic environment internally of the sheath 30. Protective sheath 30 may preferably be a one-piece, seamless enclosure or may be multiple sheets such as top and bottom sheets that are sealed along their facing edges. With continuing reference to FIG. 3, the sheath 30 is attached to the attachment flap 32 at the hand end 18 of applicator 12. The sheath 30 extends completely around and is non-detachably sealed to the attachment flap 32 as illustrated with cross hatching in the drawing adjacent the hand-receiving end 18 to define a non-detachable seal 34 that hermetically encases the entire applicator 12 within the sheath 30. It will also be noted that an optional adhesive may be applied to the facing inner surfaces of inner liner 22 around the opening 16 into cavity 14—that is, the facing surfaces of attachment flap 32—the adhesive allows the user to separate the facing surfaces so that the user's hand may be inserted into the cavity. As shown in particular in FIG. 9, the upper and lower layers of sheath 30 are releasably sealed together along the aligned edges of protective sheath 30 at the end 20, opposite of hand-receiving end 18, at a releasable, detachable seal 36, referred to at times herein as the protective sheath releasable seal 36. The seal 36 between the upper and lower sheets of sheath 30 at seal 36 will separate with the application of force as detailed below.

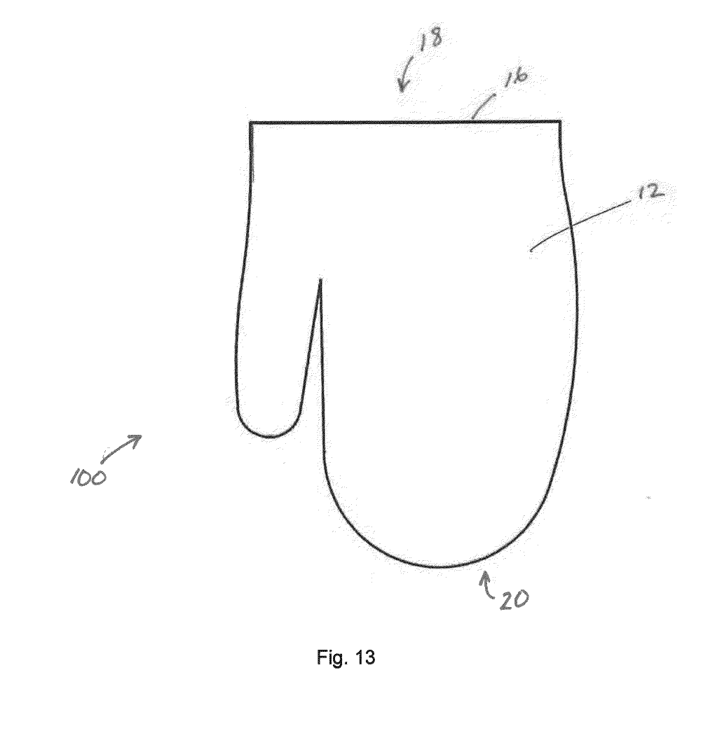

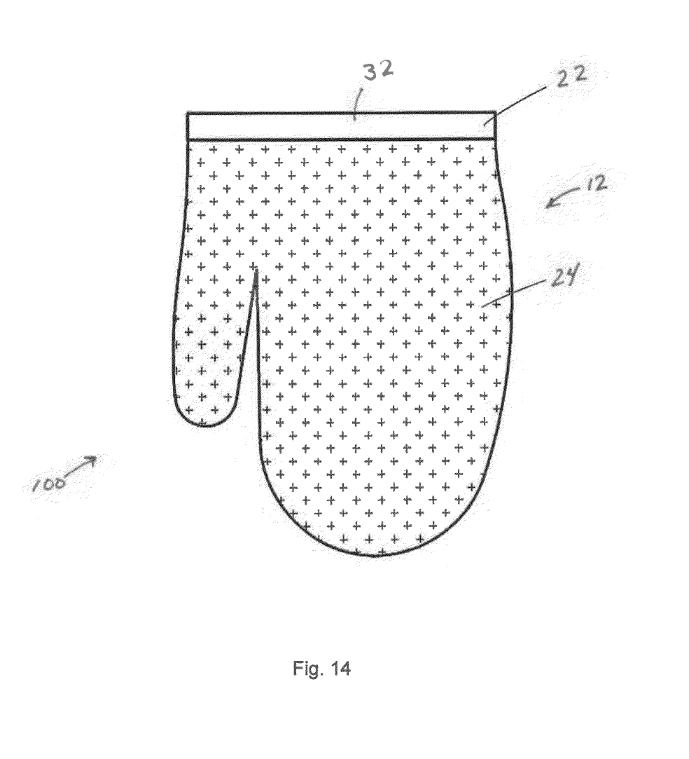

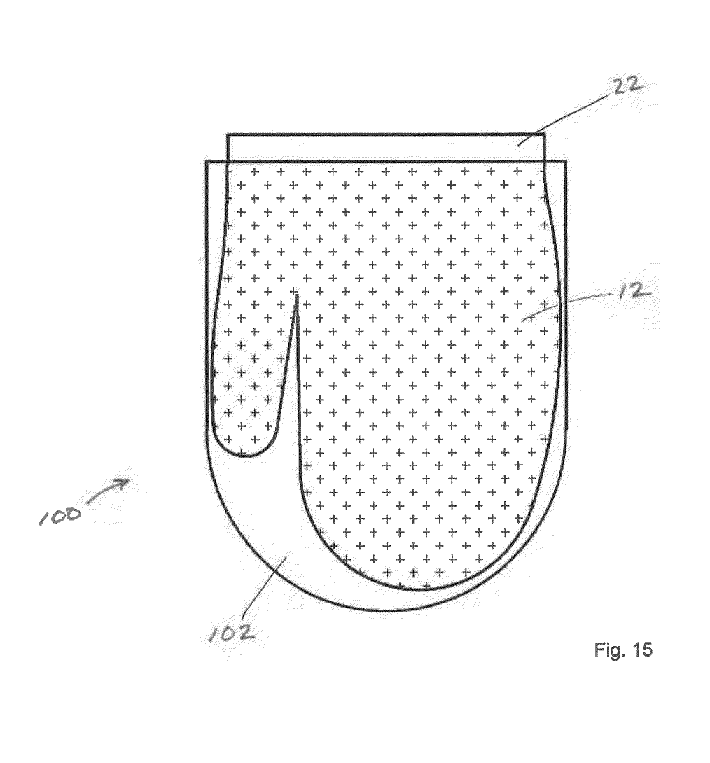

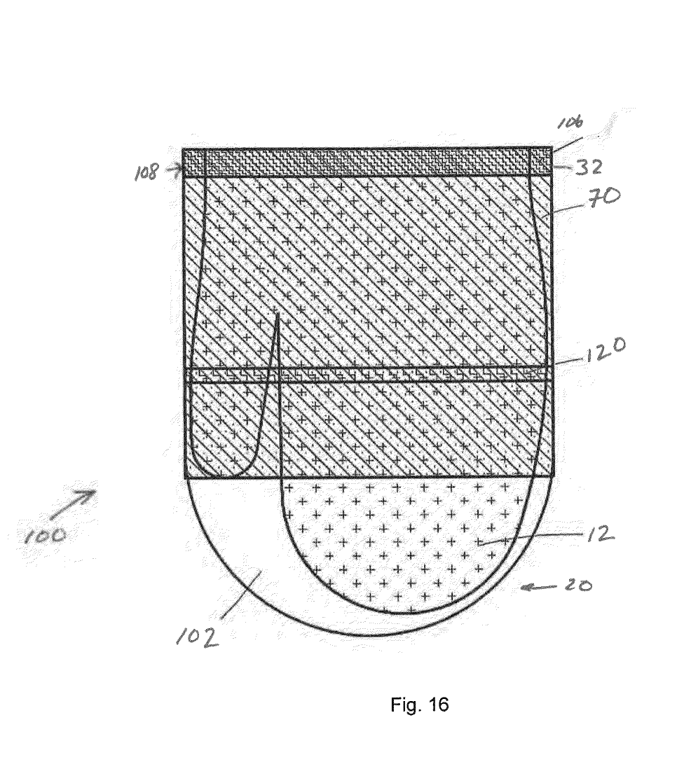

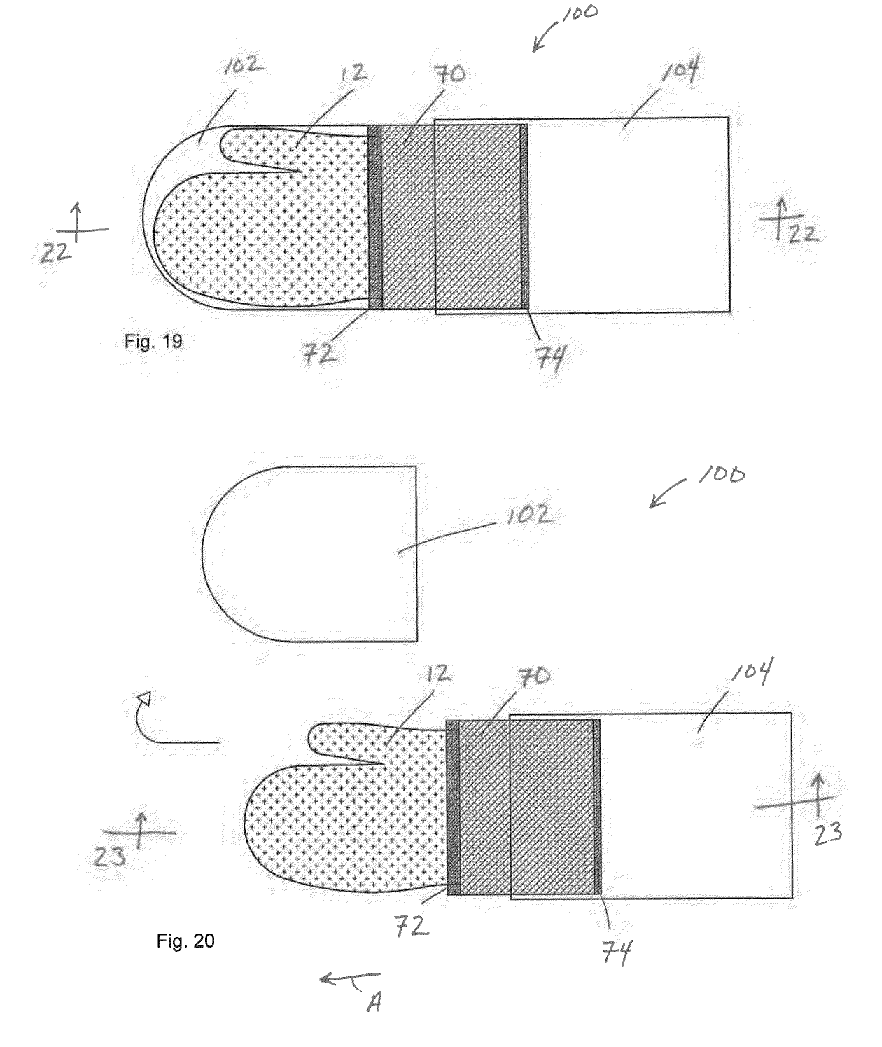

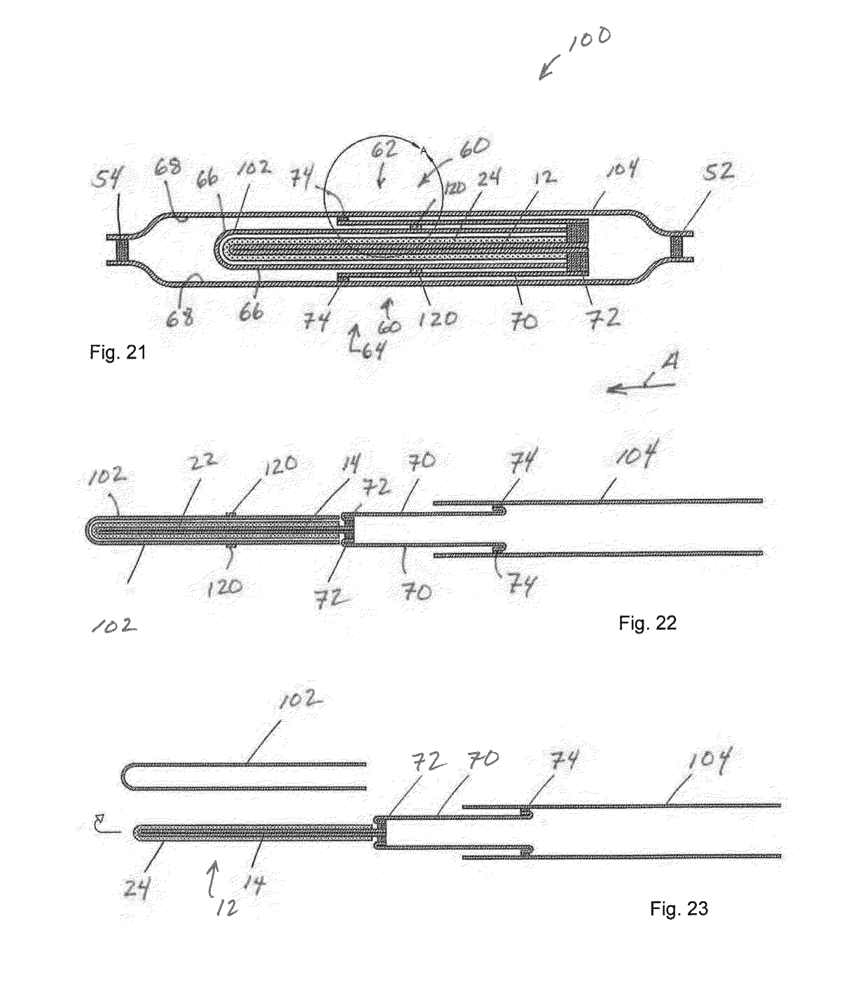

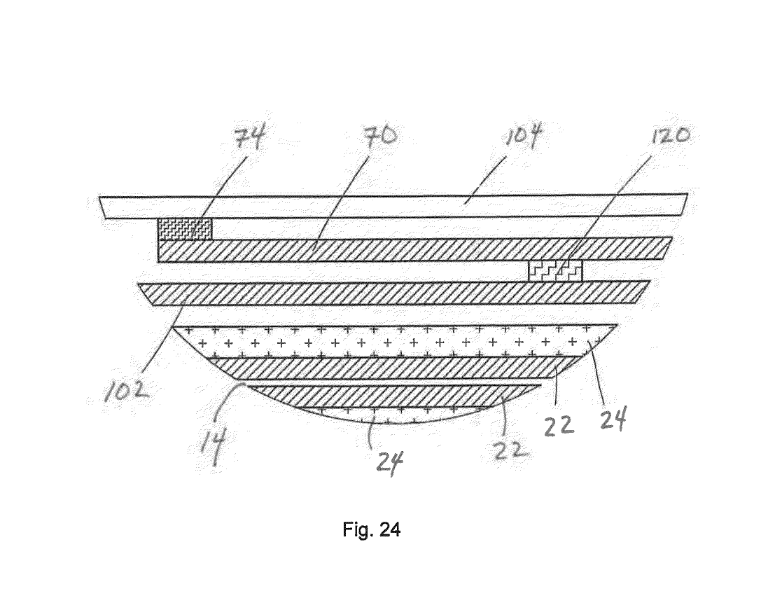

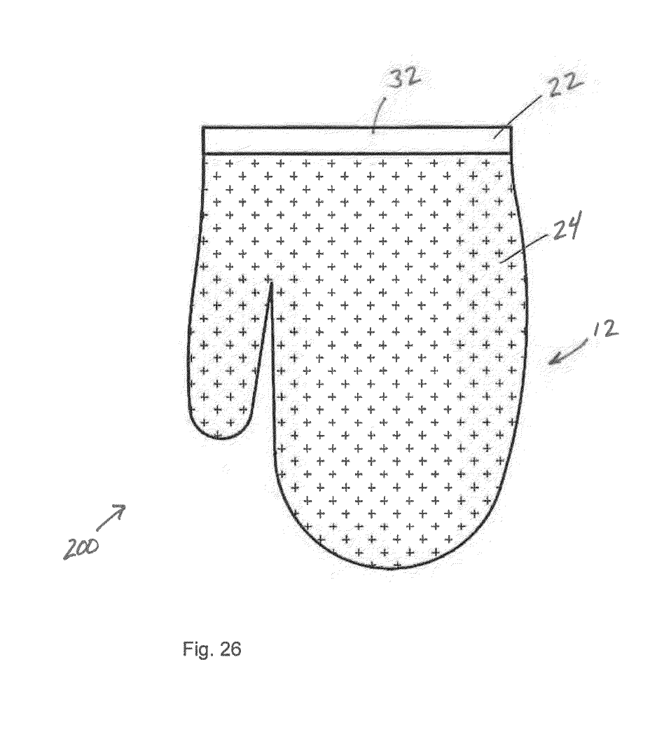

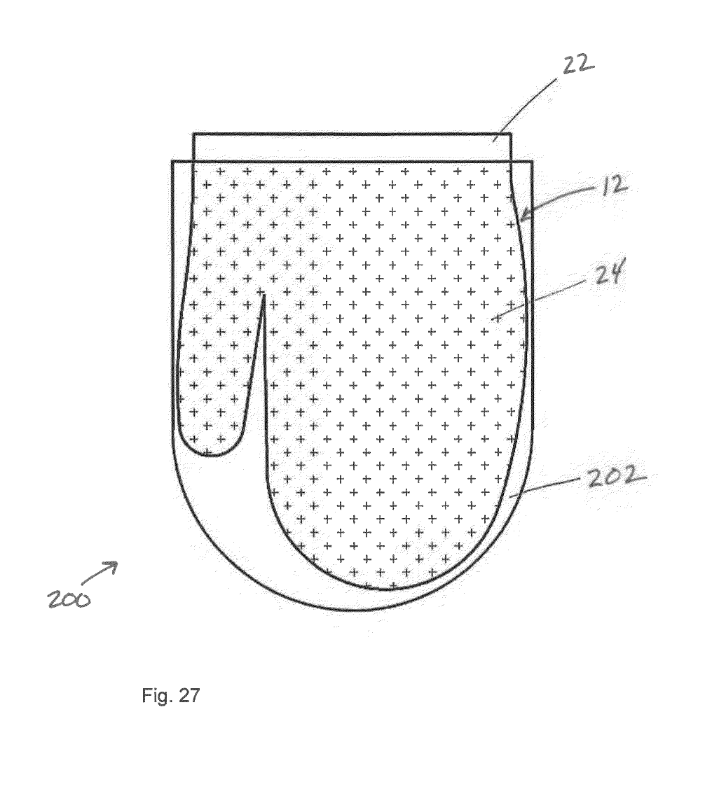

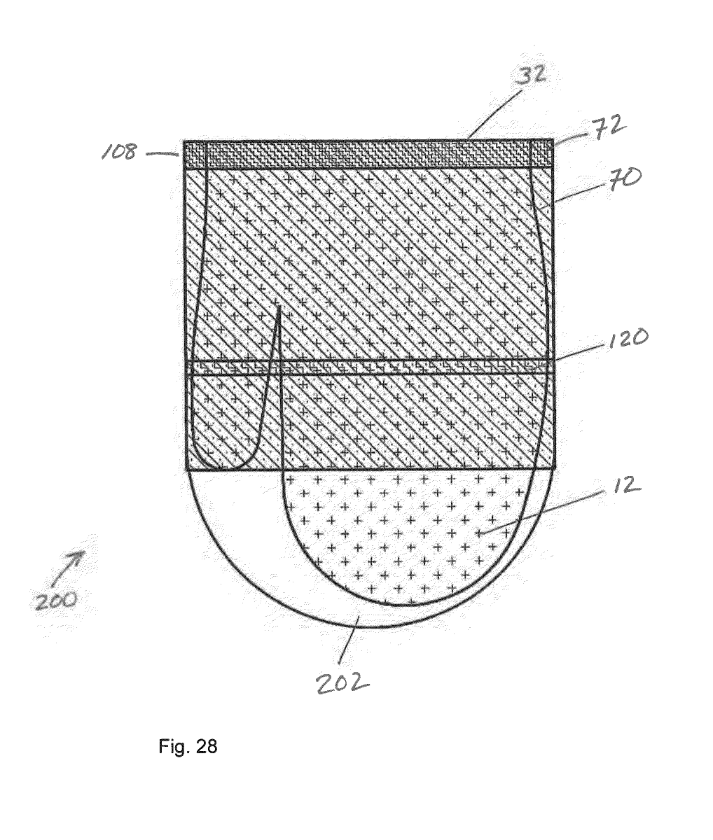

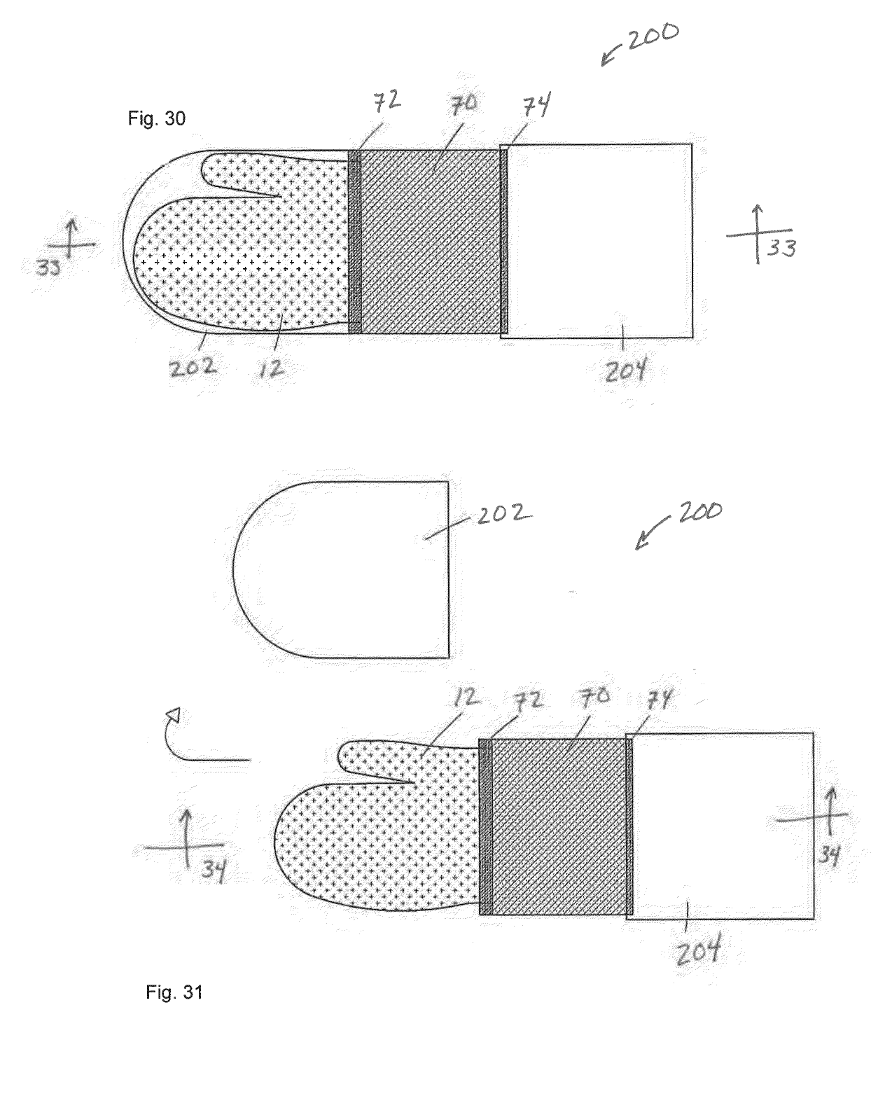

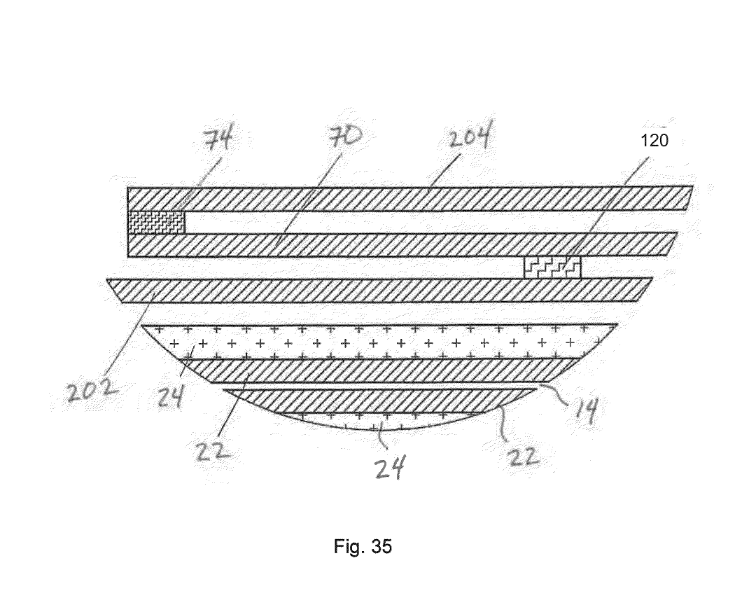

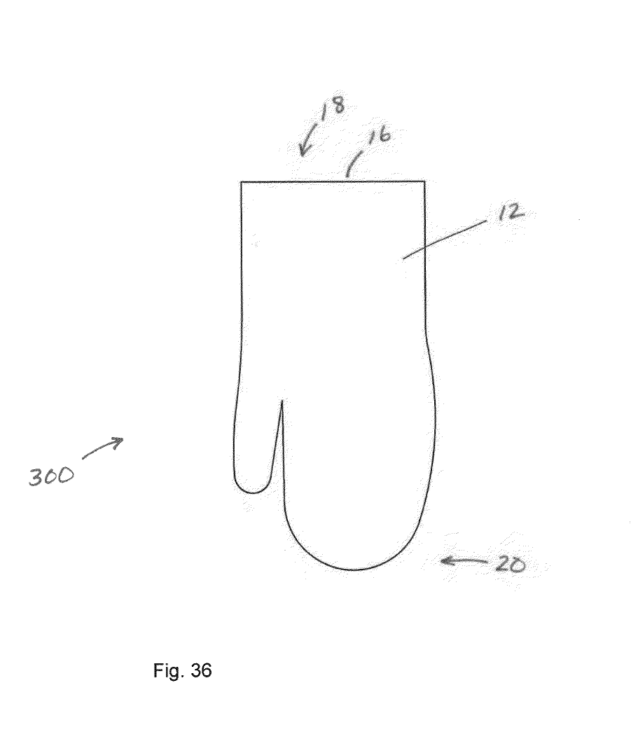

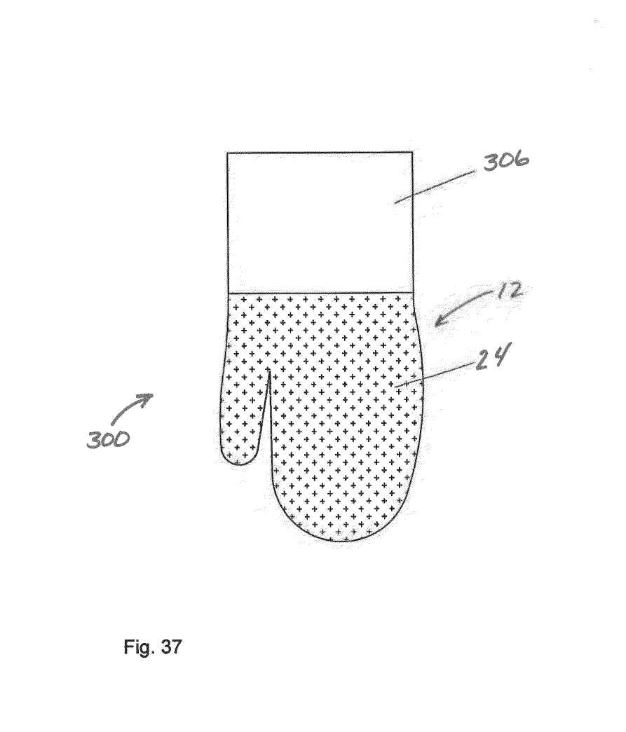

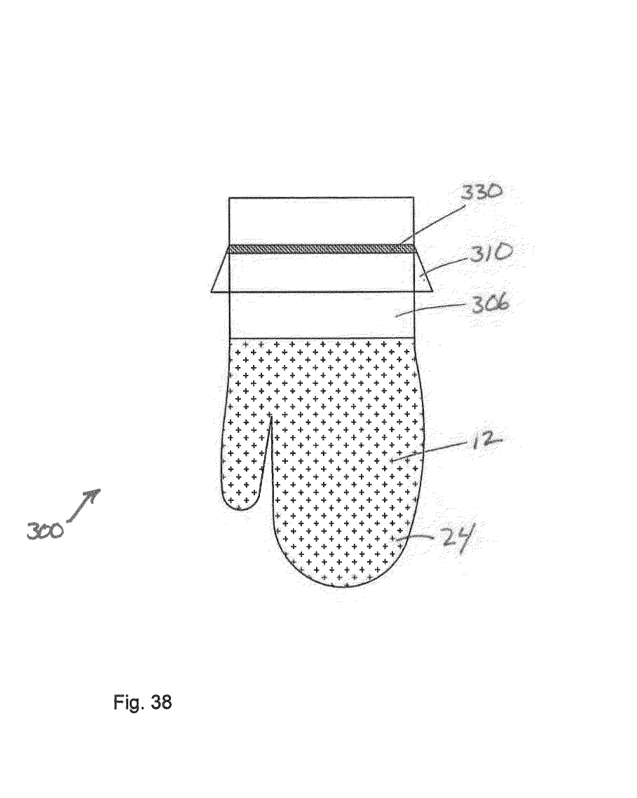

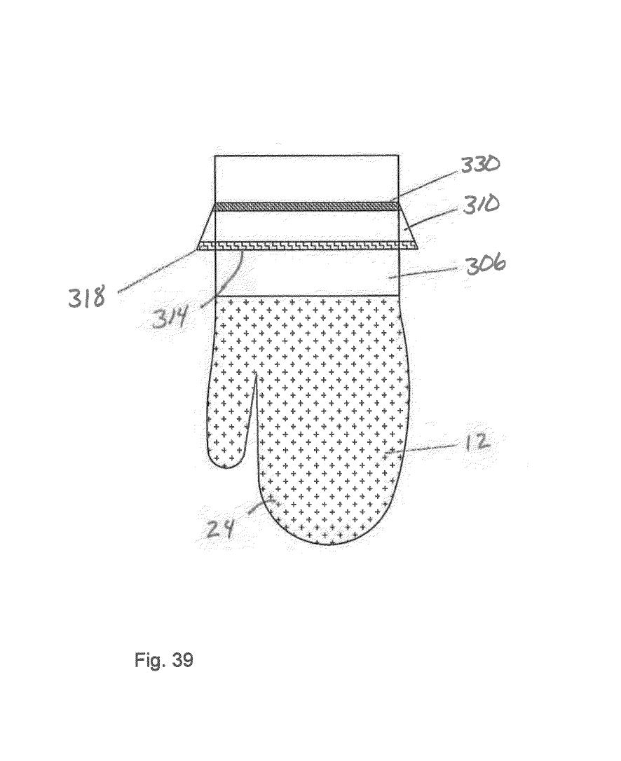

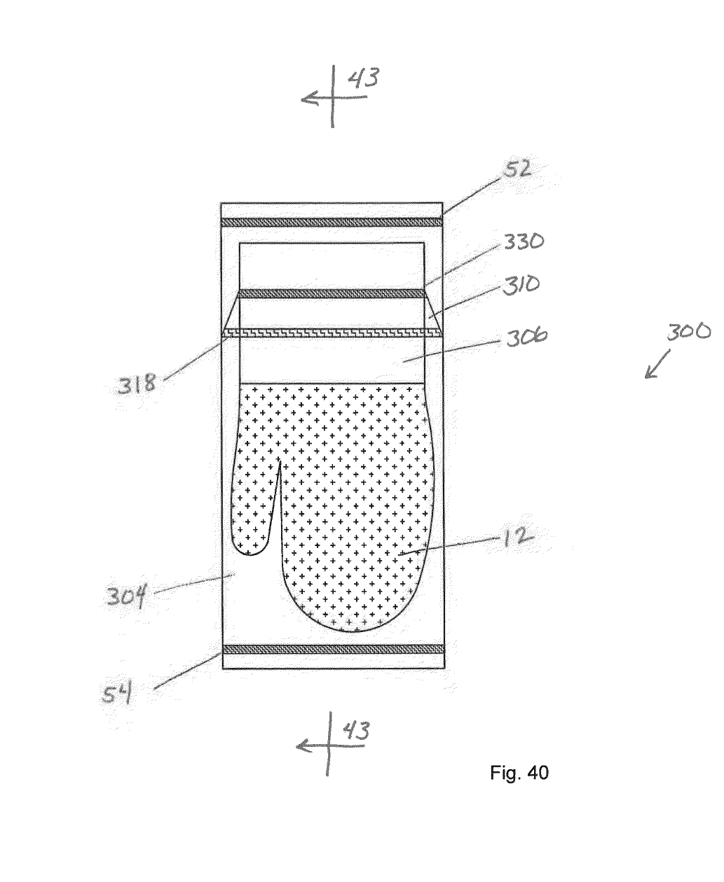

In other embodiments detailed below, for example, the embodiment illustrated in FIGS. 13 through 24 and FIGS. 25 through 35, the external sheath of the protective enclosure is completely removable from the applicator 12. In such embodiments, the external sheath is defined by a seamless bag-like covering that extends over the outside surface of the applicator 12. Instead of attaching to the attachment flap 32 of the liner 22, the external sheath remains free for easy removal to expose the encased applicator 12 for use. The aseptic environment is maintained, however, by an arrangement of seals, described below, that effectively close off the applicator 12 within the aseptic conditions of the protective sheath.

The various sheets making up the protective sheath and the various seals described herein can be attached together, as described above, by any number of sealing methods known in the art. For example, by thermal bonding, radio-frequency welding or bonding, sonic welding or bonding, adhesive bonding (using any of the number of adhesives including but not limited to spray adhesives, hot melt adhesives, latex-based adhesives, water-based adhesives, and the like). As will be apparent to those skilled in the art, the sealing method used will be compatible with, and able to withstand, sterilization methods.

### External Sheath

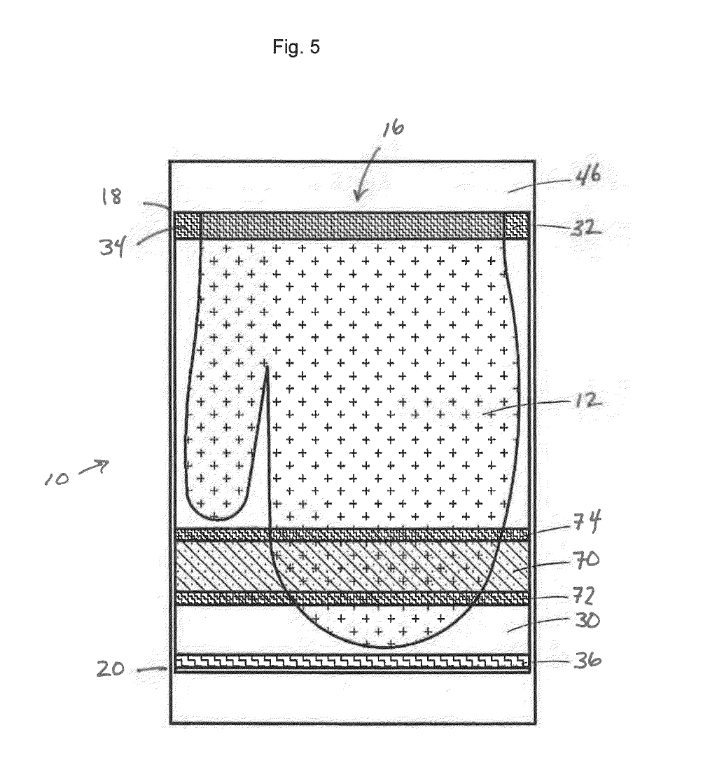

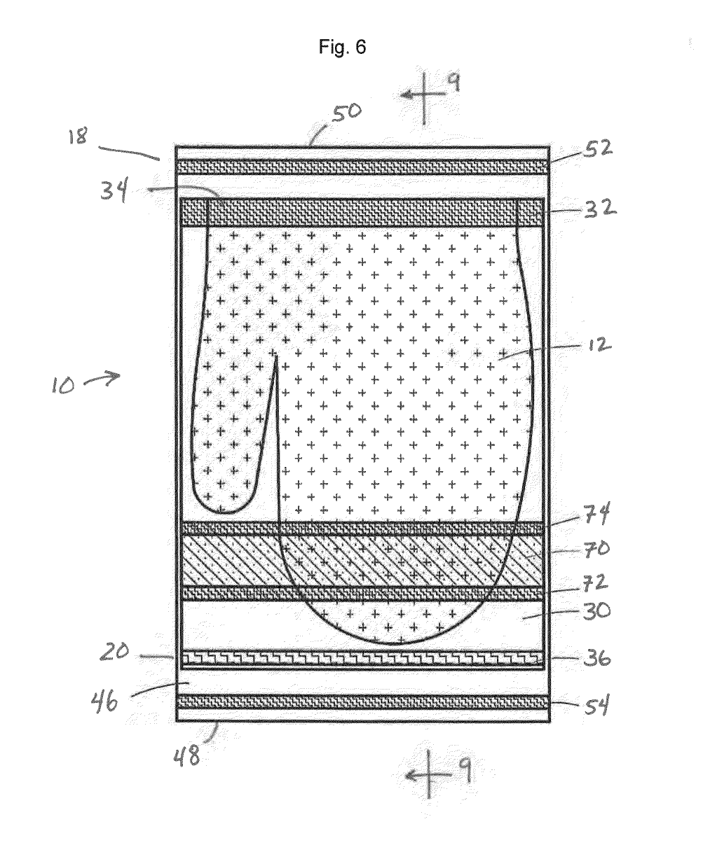

Continuing now with the description of the embodiment of FIGS. 1 through 12, and more specifically with reference to FIG. 5, the combination of applicator 12 and protective sheath 30 are at least partially contained within an external sheath 46. The external sheath 46 shown in FIG. 5 is closed at the end 48 and open at the end 50 adjacent hand-receiving end 18 so that the opening 16 to internal cavity 14 is open. The applicator 12 is nonetheless maintained in the sterile field internally within protective sheath 30 by the seal between the sheath 30 and the attachment flap 32 as detailed above. In the embodiment shown in FIG. 6, the external sheath 46 is sealed adjacent both ends of the applicator 12, namely, at seal 52 adjacent end 50 (the “proximal end”) and at seal 54 adjacent the opposite end 48 (which is the “distal end”). The seals 52 and 54 preferably define a completely aseptic enclosure for applicator 12 and its protective sheath 30 and thus provide the external packaging for the self-contained skin prep system 10. The seals 52 and 54 may be broken to open the sheath 46 at both ends at the time of use. In such embodiments, the external sheath 46 may be labeled to include product information and/or instructions for use directly on the outside surface of the external sheath 46.

### Deployment System

The skin prep system 10 according to the present disclosure utilizes structures that are part of a deployment system and which allow the applicator 12 to be maintained in an aseptic condition during storage, while enabling rapid aseptic deployment of the applicator when needed, unassisted, by a user. Deployment of the system is detailed below, after the structures that facilitate deployment and sterile storage are described. Referring to FIGS. 3 through 12, the deployment mechanism is referred to generally with reference number 60 and comprises a cooperative arrangement of the protective sheath 30, external sheath 46, and a pair of restrictors 62 and 64 that allow the pre-loaded applicator 12 to be deployed from the protective sheath in a controlled manner while maintaining aseptic conditions and practices. When the applicator 12 is in the deployed position, the protective sheath 30 is separated from the applicator to expose the applicator for use in scrubbing a target location on a patient.

As detailed above, prep delivery system 10 comprises a protective sheath 30 that encases applicator 12 in a sealed, aseptic field within the sheath and an external sheath 46 that encases the protective sheath 30. As shown in FIG. 6 and as described above, the seals 52 and 54 at the respective opposite ends 50 and 48 of the protective sheath 46 are releasable types of seals that may be broken by the users, which allows the user to insert his or her hand into the opening 16 and thus into cavity 14 of the applicator 12.

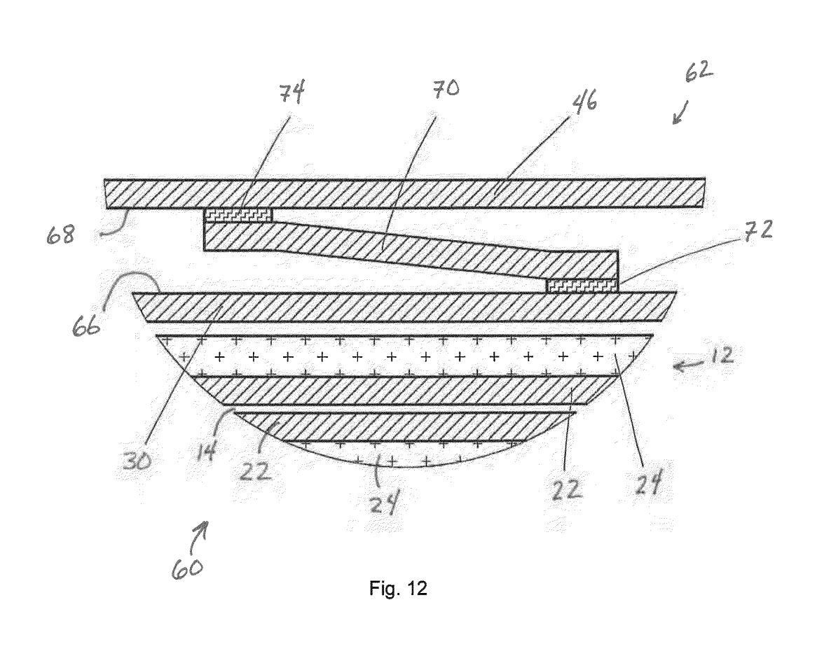

The seals 52 and 54 are formed as noted above by any appropriate method such as thermal bonding, RF welding, sonic welding or bonding, adhesive bonding. These seals are impermanent and may be broken by the user by pulling the opposite layers of the sheath 46 apart. Once a user has released or broken the seals, the opening 16 and hand end 18 is exposed and the user may insert his or her hand into the applicator 12. The applicator 12 may continue to be extended through the length of the protective sheath 30 and external sheath 46 by the user pushing her hand into the applicator 12 in the direction of arrow A in FIG. 8 in order to fully expose the applicator 12 for use. The extent to which the applicator 12 can be extended in the direction of arrow A through the protective sheath 30 and external sheath 46 is controlled and limited by the deployment system 60, and more specifically by one or more restrictors 62, 64 that attach between the outer surface of the protective sheath 30 and the facing, inner surface of the external sheath 46. The deployment system 60 is best illustrated in the detailed drawing of FIG. 12 where it may be seen that a restrictor strip 70 is attached at one 72 end to the outer surface 66 of protective sheath 30 and at its opposite end 74 to the facing, inner surface 68 of external sheath 46. The attachments at the ends 72 and 74 define a restrictive, but ultimately detachable connection between the restrictor strip and the surfaces to which it is attached. More specifically, the restrictor strips initially limit the relative translational travel between the applicator, the protective sheath and the external sheath—the strips allow the protective sheath to extend out of the external sheath in the direction of arrow A, and the strips ultimately assist in the applicator breaking through the seal. But the connection between the restrictor strips and the external sheath may be broken to separate the external sheath. The restrictor strip 70 is of a defined width between its opposite ends that are attached to the respective outer and inner facing surfaces, and that width determines the distance to which the applicator 12 may be extended relative to the protective sheath 30 and external sheath 46. As best shown in, for instance, FIG. 9, each skin prep delivery system 10 utilizes two restrictor strips 70, one on each of the opposite sides of the applicator 12 as shown.

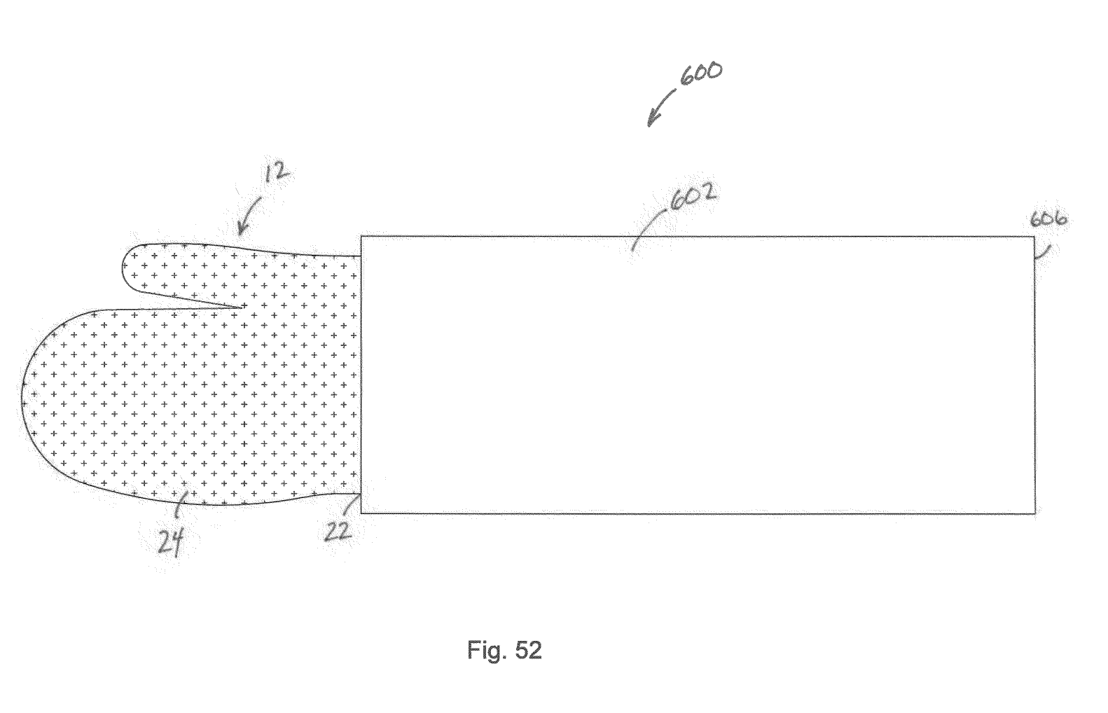

As detailed above, applicator 12 is at all times prior to deployment of the system 10 held in an aseptic enclosure within protective sheath 30. Ideally, external sheath 46 similarly defines an aseptic enclosure in the space between the external sheath and the protective sheath 30. However, in some instances and for a variety of reasons it may be difficult to insure sterility in the space between the protective sheath 30 and the external sheath 46. Accordingly, structures have been designed to insure sterility of the applicator as it is deployed, as detailed below in connection with FIGS. 52 through 58.

Specific attention is now made to the series of sectional view of FIGS. 9, 10 and 11. In FIG. 10, skin prep delivery system 10 is shown in its fully sealed and non-deployed condition such as when the product is being stored. In this condition, the applicator 12 is encased in the sterile enclosure defined by protective sheath 30 and all of the seals are intact—i.e., seals 52, 54 are intact and the protective sheath releasable seal 36 at aligned edges of the protective sheath 30 at end 20 and the seal 34 at hand end 18 are intact. Each restrictor strip 70 is either permanently or detachably attached at its opposite ends to the respective outer surface 66 of protective sheath 30 and the facing inner surface 68 of the external sheath 46. In the storage position shown in FIG. 9, the deployment system is shown in its non-deployed, non-extended position and the applicator 12 is centrally maintained within the external sheath 46 by the paired deployment systems 60. The skin prep delivery system 10 shown in FIG. 6 is also seen in its non-deployed, stored condition.

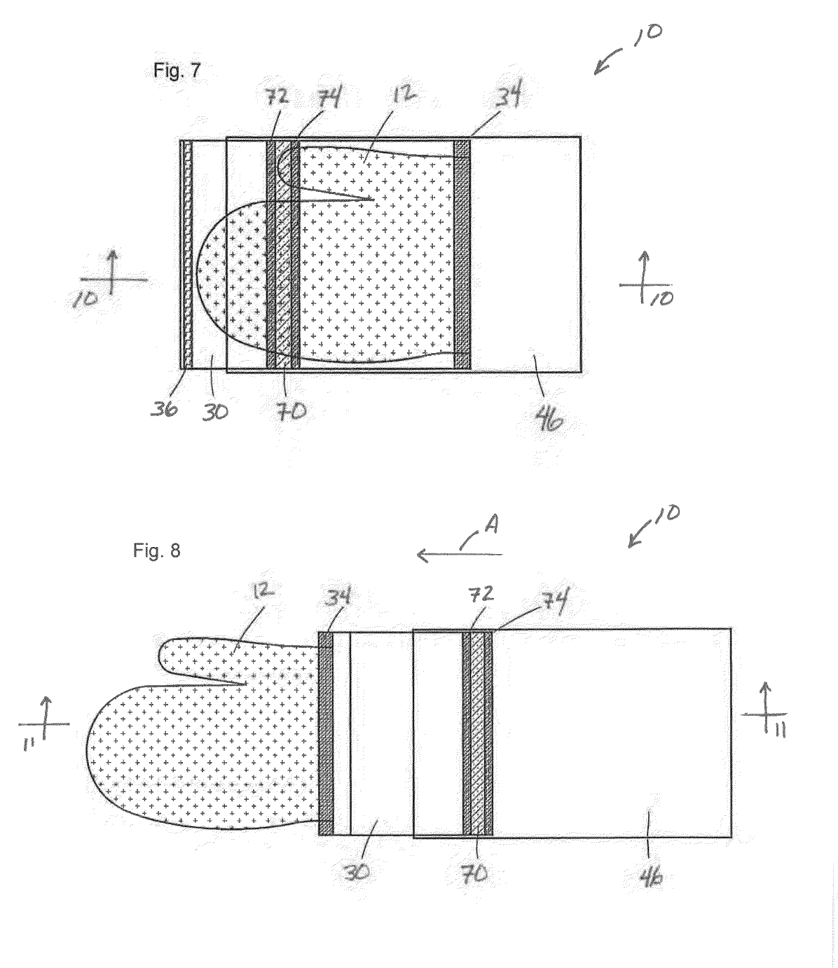

Turning to FIG. 10 and its corresponding FIG. 7, applicator 12 has been deployed to a first position of deployment. In these figures, the seals 52 and 54 of external sheath 46 have been broken, released, to thereby expose the hand end 18 of applicator 12. The applicator 12, which is still encased within the protective sheath 30 may then be extended relative to the external sheath 46, but the distance that the protective sheath 30 and the encased applicator 12 may be extended relative to the external sheath 46 are limited by the restrictor strips 70. Specifically, as shown in FIG. 10, the combination of the protective sheath 30 and the enclosed applicator 12 (still sealed at 32, 34) may be slidably translated relative to the external sheath 46, causing each restrictor strip 70 to invert until continued movement is restricted by the width of the restrictor strips. In this way, the applicator 12 may be extended relative to the external sheath 46 to a distance approximately double the width of the restrictor strips 70. And as noted previously, one the system is fully deployed the external sheath 46 may be detached and separated from the other components.

Full deployment of skin prep delivery system 10 is shown in the pair of FIGS. 11 and 8. Here, the protective sheath releasable seal 36 has been broken. The user's hand is pushed into the opening 16 and the seal 36 is broken by the user forcibly pushing her hand in the direction of arrow A so that the applicator 12 is forced against seal 36 to thereby cause the seal to rupture. Once the seal 36 is broken, the applicator 12 is released from the protective sheath 30 and may be fully extended by inversion of the protective sheath 30 until continued extension is restricted by the restrictor 60. In this fully deployed position, the sheath is separated from the applicator 12, which is fully exposed and ready to be used to disinfect the target site. It will also be evident from FIG. 11 that when the applicator 12 is fully deployed as shown, the space between the restrictor strips 70 and the distal end edges of the external sheath 46 act as an effective trap or trough for containing excess antiseptic that flows from the applicator during use. This can help prevent the antiseptic from flowing onto the external surfaces of the external sheath during use. It is further possible to include absorbent materials in the space between the restrictor strips 70 and the distal end edges of the external sheath 46, such as sponges and the like to absorb excess antiseptic.

A first alternative embodiment of a skin prep delivery system is shown in the series of FIGS. 13 through 24. In FIGS. 13 through 22 and in the discussion that follows, like structures are assigned the same reference numbers used above and in FIGS. 1 through 12.

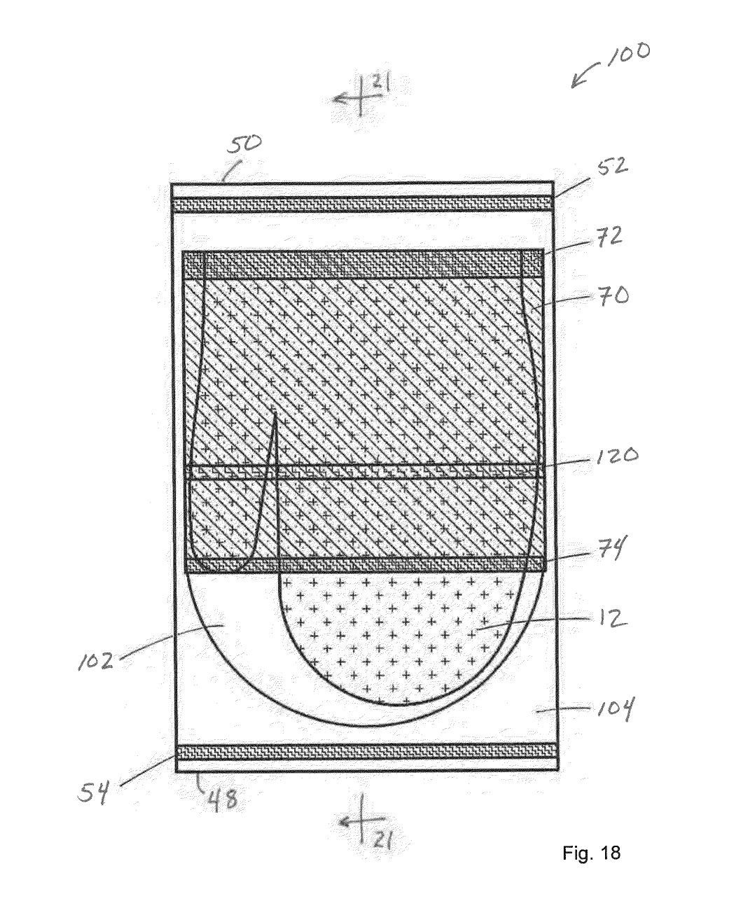

With reference to FIGS. 13 through 24, a skin prep delivery system 100 that incorporates a protective sheath 102 that is removable from the skin prep system 100 when in the deployed state to expose the applicator 12 for use is illustrated. In this embodiment, the protective sheath 102 is defined by a seamless bag-like covering that extends over the outside surface of the applicator 12 and which in a non-deployed state (e.g., FIG. 18), maintains a sterile enclosure for applicator 12. However, when the system 100 is deployed the sheath 102 is easy removed.

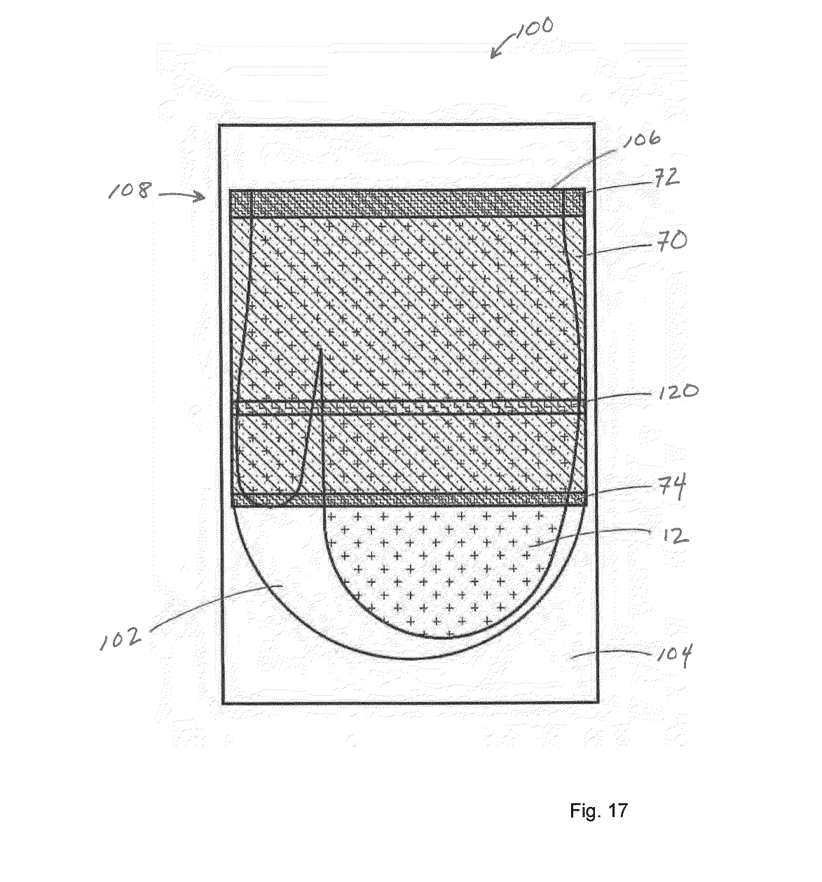

Once a user inserts a hand through the hand-receiving end 18 of the applicator 12 and into cavity 14 (separating the opposed surfaces of the inner liner 22 if an optional adhesive is used between the layers as described previously), the applicator 12 may be deployed by extending the applicator 12 through the protective sheath 101. As the applicator 12 is extended in the direction of arrow A (e.g., FIGS. 17, 20) the breakable seals 120 between the bag-like protective sheath 102 and the restrictor strips 70 are broken to release the applicator 12 from the stored position (e.g., FIG. 18). As the applicator 12, still encased in an aseptic condition by the bag-like protective sheath 102, continues to be extended through the external sheath 104 the deployment mechanisms 60 invert allowing the applicator 12 to be fully extended into the deployed position shown in FIGS. 20 and 23. It will be appreciated that as shown in FIGS. 17 and 20, the bag-like protective sheath 102 allows the applicator 12 to be fully deployed but remain encased in an aseptic environment defined by the protective sheath 102 until the user is ready to disinfect the target site, at which time the bag-like protective sheath 102 can be removed to expose the applicator 12 for use as shown in FIGS. 20 and 23.

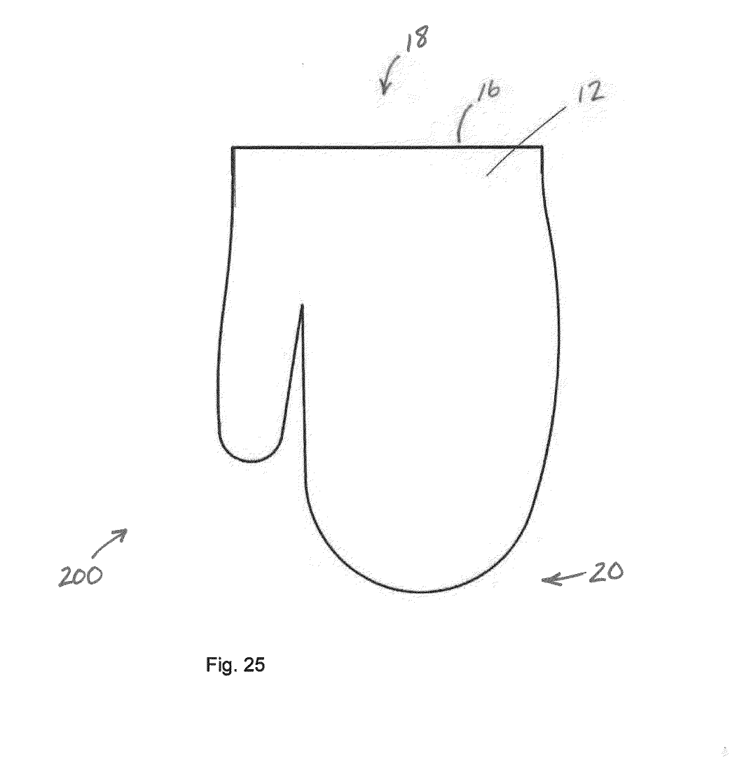

A second alternative embodiment of a skin prep delivery system 200 is shown in the series of FIGS. 25 through 35. Following the numbering and naming convention established above, like structures are assigned the same reference numbers and names used above in respect of the other embodiments.

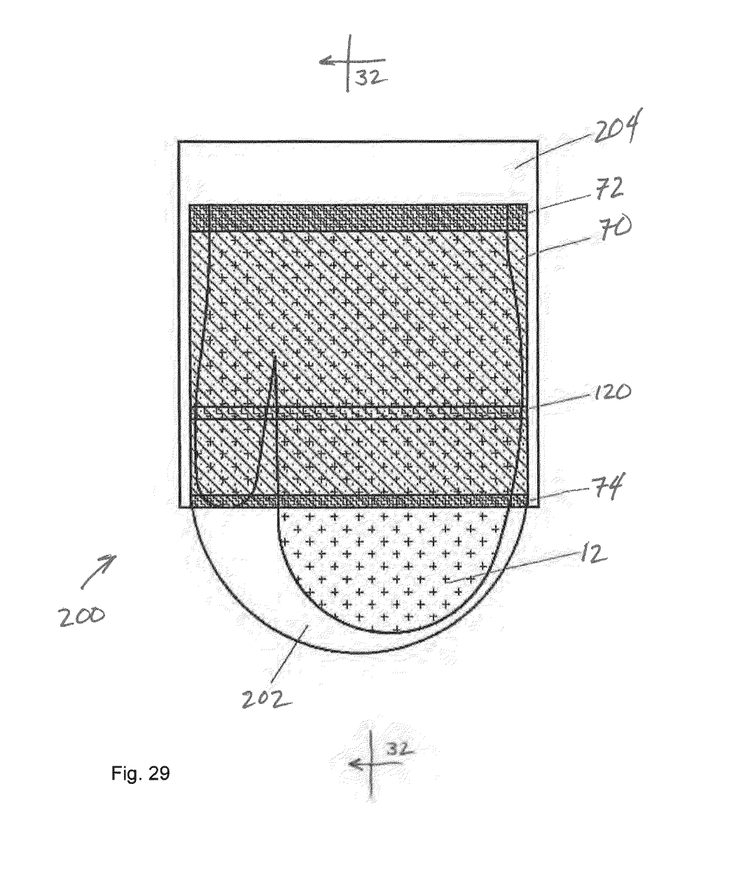

The skin prep delivery system 200 of FIGS. 25 through 35 is similar in many respects to the embodiment of the skin prep delivery system 100 of FIGS. 1 through 12 insofar as the system 200 incorporates a protective sheath 202 that is removable from the skin prep system 200 when in the deployed state to expose the applicator 12 for use. In this embodiment, system 200, the protective sheath 202 is defined by a seamless bag-like covering that extends over the outside surface of the applicator 12 and which in a non-deployed state (e.g., FIG. 29), maintains a sterile enclosure for applicator 12. When the system 200 is deployed the sheath 202 is easily removed.

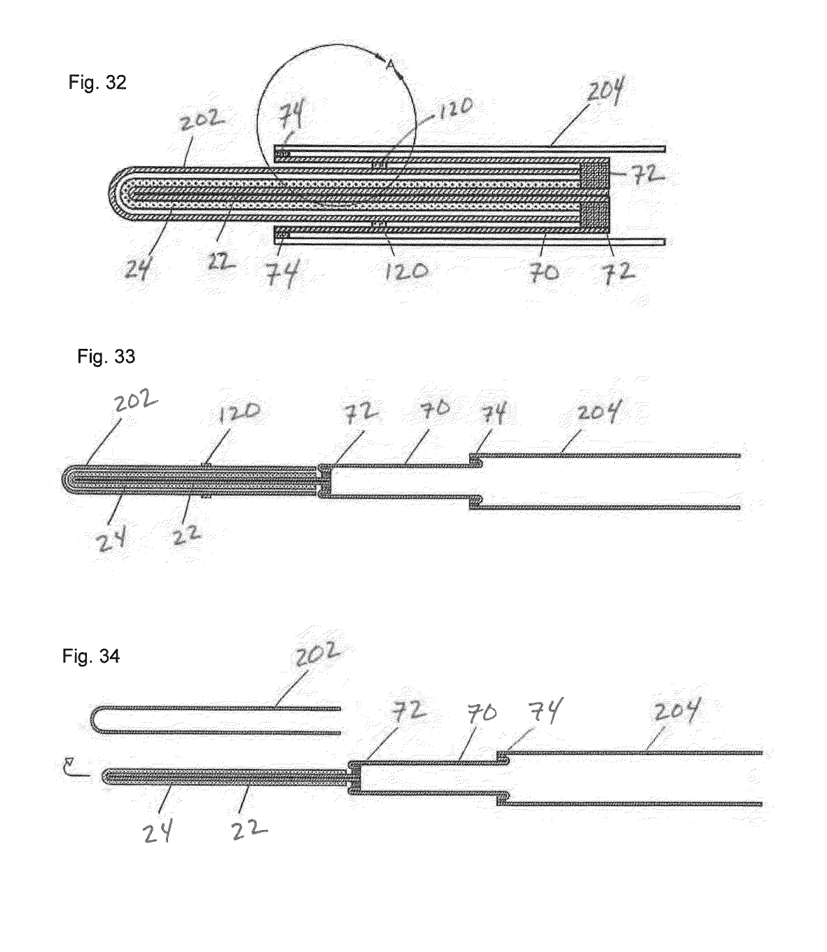

The skin prep delivery system 200 incorporates a different arrangement of restrictor strips 70 with different relative positions from those used in prior embodiments. With reference to the series of cross section views of FIGS. 32, 33 and 34 and the corresponding plan views of 29, 30 and 31 it may be seen that when system 200 is in the non-deployed storage state (e.g., FIGS. 29, 32) the external sheath 204 encases protective sheath 202 only partly along the length of protective sheath 202 and defines a seal between the external sheath and the protective sheath at the intermediate attachment 120. Because the combined protective sheath 202 and external sheath 204, coupled with the seals defined by intermediate attachment 120 and at ends 72 and 74. Accordingly, the seals 52 and 54 may be and are omitted in this embodiment 200.

Yet another embodiment of a skin prep delivery system 300 is illustrated in the series of figures of 36 through 46. The system 300 of this embodiment defines a hermetically sealed aseptic environment that is provided by the external sheath 304 wherein the protective sheath described above with respect to prior embodiments is eliminated and an extended portion of applicator 12 defines an elongated sleeve 306 that extends in its deployed state.

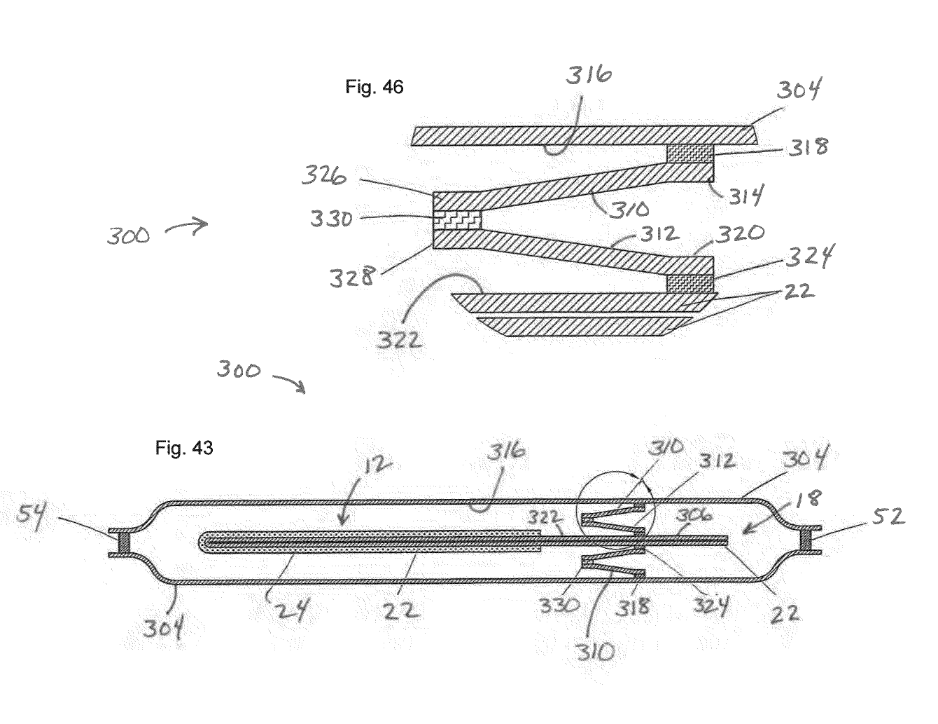

System 300 utilizes a pair of restrictor strips 310, 312 that are releasably connected to one another to define a breakable seal. With specific reference to the sectional views of FIGS. 43 through 46 it may be seen that restrictor strip 310 has its first end 314 non-detachably attached to the inner-facing surface 316 of external sheath 304 at a non-breakable seal 318 therebetween. Similarly, restrictor strip 312 has its first end 320 non-detachably attached to the outer-facing surface 322 of inner layer 22 of applicator 12, which is exposed in this embodiment to provide an area for a non-breakable seal 324 between the first end 320 and the layer 22. The respective second ends of restrictors 310 and 312, labeled with reference numbers 326 and 328 are releasable interconnected at a seal 330. Each end of the external sheath 304 is sealed closed as with breakable seals 52, 54, thereby enclosing the applicator 12 within what ideally is a hermetically sealed environment. FIG. 43, for example.

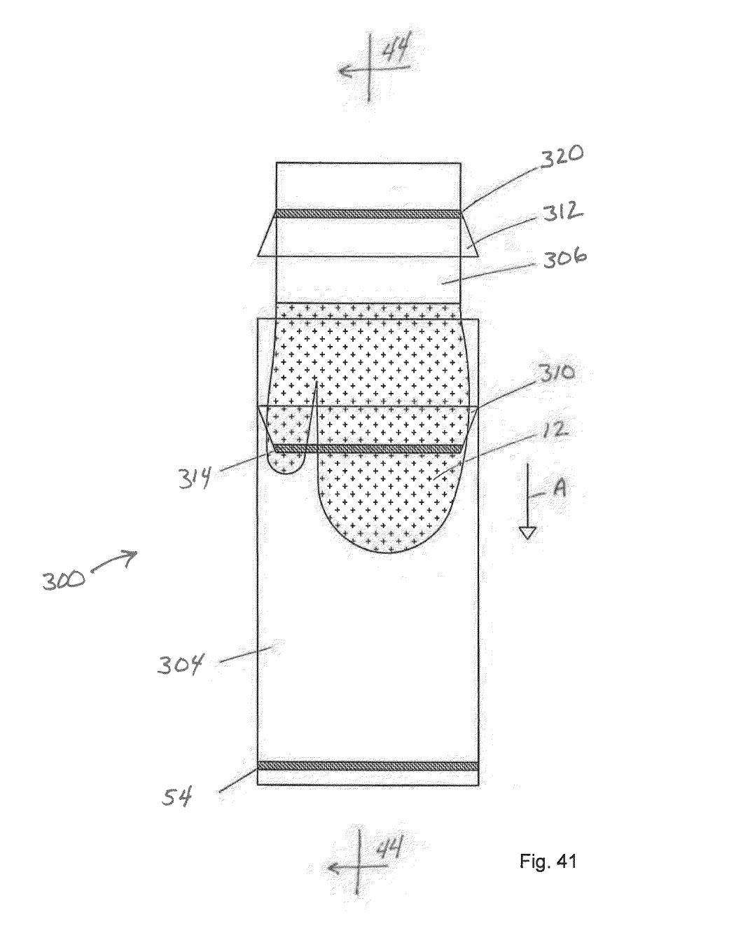

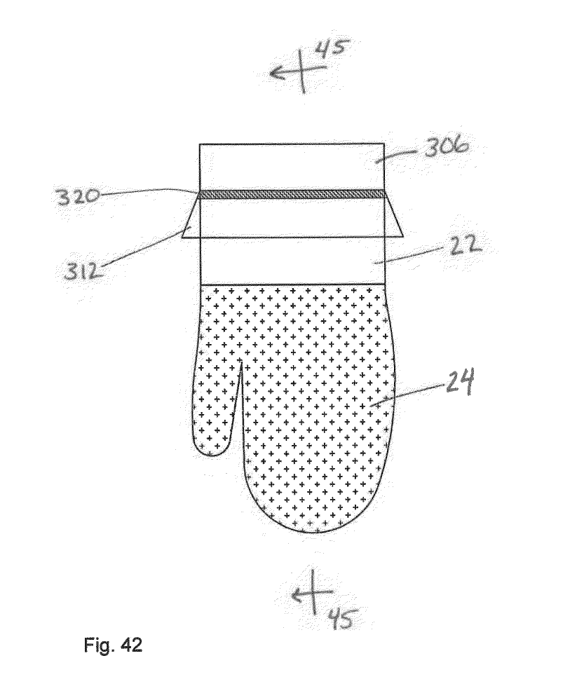

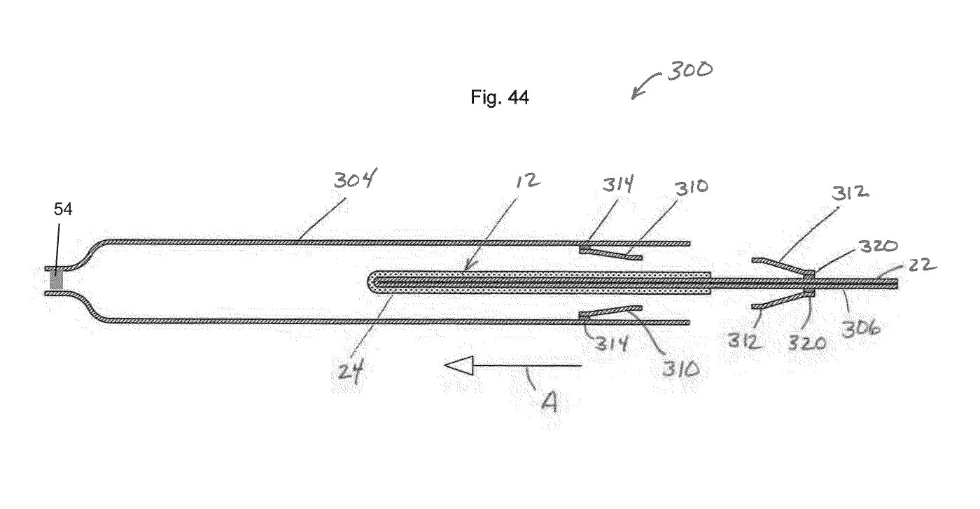

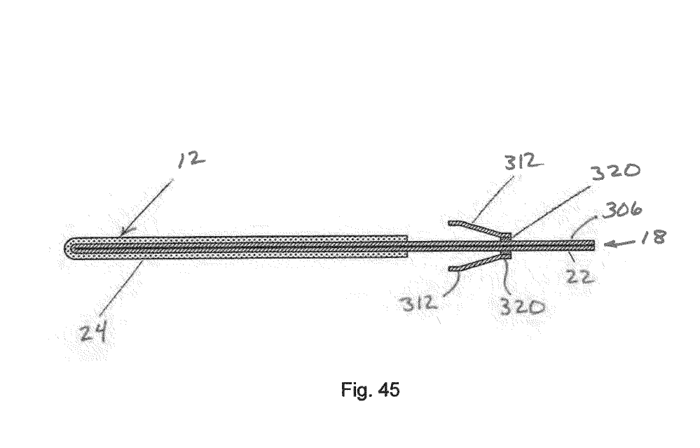

Restrictor strip 310 of the pair 310, 312, provides an anchor to the external sheath 304 while the other restrictor 312 of the pair provides an anchor to the inner layer 22 of applicator 12. It will be appreciated that the restrictor strip 312 may also function as an antiseptic trap or barrier. The applicator 12 is in this embodiment centrally maintained within the external sheath 304 when in the stored, non-deployed position. When in a first position of deployment, as shown in FIG. 41 and the corresponding FIG. 44, the seal 52 between the ends of the outer sheath 304 is broken open to allow a user to insert a hand through the hand-receiving end 18 of the applicator 12. The external sheath 304 may then be pulled or peeled away from the applicator 12, slidably translating away from the applicator 12 and causing one of the pair of restrictors 310, 312 to invert until continued translational movement (arrow A, FIG. 44) is restricted. Continued pulling away of the external sheath 304 from the applicator 12 beyond the first position of deployment results in breaking of the seal 330 between the second ends 326 and 328 of restrictor strips 310 and 312. Once the seal 330 is broken, the applicator 12 is released from its attachment to the external sheath 304 and the applicator 12 may be fully deployed as shown in FIGS. 42 and 45 and ready for use to disinfect the target site.

As detailed above in respect of the various embodiments, (e.g., FIGS. 8, 20, 31), when the skin prep system is deployed for use, the applicator 12 is extended through the external sheath of the particular embodiment (e.g., in respect of FIGS. 8, 20, 31, external sheath 46, 104, 204) while the external sheath slides over the user's forearm. In this way, the external sheath operates as an extension of the applicator 12, helping to secure the applicator 12 to the user's hand. Once a patient's skin has been prepared as desired with applicator 12, the used applicator 12 may be easily withdrawn back inside the external sheath thereby providing a compact and enclosed package for disposal.

In yet other embodiments, (e.g., the embodiment of FIG. 44), when the skin prep system 300 is deployed for use, the external sheath 304 is pulled away from the applicator 12 to expose the applicator for use. In such embodiments, the extended sleeve 306 of the applicator 12 extends over the user's forearm to define an arm-covering sleeve that assists in securing the applicator 220 to the user's hand and protects the user's arm.

With reference to FIG. 47, an alternative embodiment defining an alternative method for delivering antiseptic to applicator 12 is disclosed. In this embodiment a predetermined and desired volume of antiseptic preparation is contained in a separately sealed ampule 150 that is retained within the external sheath of the particular embodiment (not shown in FIG. 47—it will be understood that this method of delivering antiseptic to agent application material 24 on applicator 12 may be used with any of the embodiments described herein) and held near the “palm” of the applicator 12. In the embodiment illustrated in FIG. 47, when the user desires to deliver the antiseptic preparation onto agent application material 24, the ampule 150 is squeezable to rupture the ampule to thereby release the antiseptic preparation onto the agent application material 24. The ampule may be ruptured either prior to full deployment of the applicator 12 or after the applicator has been fully deployed.

Various examples of applicators 12 having an ampule are shown in greater detail in FIGS. 47A through 47P. In these examples, the antiseptic is contained in a fluid-containing reservoir that is sealed until ready for use, at which time the reservoir is ruptured (e.g., by squeezing, puncturing, or breaking the ampule 150). As detailed below, the applicator 12 may incorporate a mechanism that allows the user to control the dispersion of antiseptic to the agent application material. The fluid-containing reservoir of the ampule, which may act as a liquid and gas barrier, may be made of one or more layers of any suitable material, such as polymers (e.g., polyesters, plastics, rubber), a film (e.g., a metalized or non-metalized film), roll stock, or glass. The application of an adhesive or binder may be used to combine layers. The rigidity of the material may be selected to achieve the desired release of fluid when the reservoir is ruptured. For example, the material may be elastically or non-elastically expandable prior to rupture, or the material may be frangible such that it cracks or breaks when a force is applied to the reservoir wall. The properties of the reservoir material may affect how fluid is released when the reservoir opens (e.g., when it is ruptured, punctured, or broken). For example, a polymer reservoir that is configured to rupture along a wall or seam may provide an initial forceful flow of fluid. In another example, a frangible (e.g., glass) fluid-containing reservoir may provide a less forceful initial flow of fluid when it is broken. Furthermore, the fluid-containing reservoir of the ampule may be selectively permeable to sterilization techniques, such as those using ethylene oxide or heat.

Various examples of ampules described herein may include one or more layers that allow fluid to pass through and that may allow a user to control the dispersion of fluid onto an agent application material. The layers may include openings, which may be in any shape, size, or form that allow fluid to pass through the material. For example, openings may be described as and may include perforations, pores, cuts, abrasions, seepageways, passageways, or any other avenue for fluid to pass from one side of the layer to the other side of the layer. Although the terms “perforations” and “pores” may be used to describe various specific examples of types of openings, it should be understood that any embodiment that refers to a layer with perforations may alternatively include a layer having pores, and vice versa. Furthermore, the same layer may include different types of openings. The openings through a layer (e.g., perforated layer 904, layer 904a having pores, layer 914, layer 916) may be any size suitable for allowing fluid to pass through. The layers may include any material described in connection with the inner lining 22, foam, felt, or a woven or non-woven material made from fibers such as cotton, pulp, rayon, polypropylene and polyethylene blends, etc. The fibers may be one or more of natural, synthetic, absorbent, or heat-sealable.

With specific reference to FIG. 47A, ampule 150 may be defined as a fluid-containing reservoir 900 that in a sealed and un-ruptured condition contains a quantity of fluid 902, such as an antiseptic solution. Ampule 150 may further comprise a perforated layer 904 that overlies (or envelopes, as described below) the fluid-containing reservoir 900. The perforated layer 904 may be positioned between the fluid-containing reservoir 900 and the agent application material 24. The fluid-containing reservoirs of any of the examples described herein may be configured to release fluid upon the opening of the reservoir, which may result from the application of pressure to the reservoir (e.g., by squeezing or puncture) or by fracturing/breaking of the reservoir.

In FIG. 47A, a compartment such as pocket 906 has been formed for receiving the fluid-containing reservoir 900. The compartment may be any enclosure configured to receive a fluid-containing reservoir 900. The compartment may be surrounded on all sides by material, and at least a portion of the wall of the compartment may include a material having a plurality of openings (e.g., perforations or pores) as described in any of the examples herein. In one example, the exterior wall of the pocket 906 may include a portion of the flexible inner liner 22 that includes openings, such as perforations 908. The perforated layer 904 therefore may be defined by an extension of flexible inner liner 22 that has plural perforations 908 and which forms an exterior wall of pocket 906. The interior wall of the pocket 906 may include an additional layer of material 903. The ends of material 903 may be fixed to the inner liner 22, as shown in FIG. 47A. Accordingly, the reservoir 900 may be captured between the material 903 and the perforated layer 904 (e.g., the portion of the inner liner 22 that includes openings), and the perforated layer 904 may be captured between the fluid-containing reservoir 900 and the agent application material 24. In an alternative example, the perforated layer 904 may include a different material than the inner liner 22 and may form the exterior wall of pocket 906, and the inner liner 22 may form the interior wall of pocket 906. Accordingly, the pocket 906 may be bounded by the inner liner 22 on the interior wall and by the perforated layer 904 of a different material on the exterior wall.

The ampule 150 may be either unattached in pocket 906, or may be attached to one or more of the inwardly-protruding layer of the inner liner 22, the perforated layer 904, or the agent application material 24. It will be appreciated that a user may insert her hand into cavity 14 and manually squeeze the fluid-containing reservoir 900 to thereby rupture the reservoir so that the fluid 902 is dispersed therefrom, as detailed below.

As an alternate to the embodiment of FIG. 47A, the ampule 150 may also be formed as a separate module, as shown in FIGS. 47B and 47C. In this embodiment, the ampule 150 is defined by a fluid-containing reservoir 900 containing fluid 902 and an outer perforated layer 904 that completely envelopes the reservoir 900 and which is sealed around its peripheral edges. This embodiment eliminates the need for a pocket 906 and contemplates that the ampule 150 be inserted in a desired location between the inner layer 22 and the agent application material 24. In both the embodiment of FIG. 47A and the embodiment of FIG. 47B there may be a fluid collecting volume, such as space 912, defined between the fluid-containing reservoir 900 and the perforated layer 904—the space 912 may be empty when the fluid-containing reservoir 900 is intact, but when the reservoir is ruptured the fluid 902 contained therein may flow into the space 912. Space 912 may inhibit excessive release of the fluid 902 (e.g., “splashing”) onto the agent application material 24 when the reservoir is ruptured by providing an area in which the fluid can return to a low pressure state. Accordingly, the presence of space 912 may be useful in providing a user with control over fluid dispersal. The fluid-containing reservoir 900 may contact one or more sides of the outer perforated layer 904 at various times. Therefore, at any given time, space 912 may extend partially around the outside of reservoir 900, and portions of the outer wall of reservoir 900 may contact or rest against the outer perforated layer 904.

In yet another alternative the ampule 150 may be located on the outer surface of agent application material 24, as seen in FIG. 47. With this embodiment, the ampule may be ruptured when the applicator 12 is in the closed and sealed packaging as described herein, or ruptured after the applicator 12 has been deployed from the packaging. When the ampule is positioned externally to the agent application material, the ampule 150 may be removed prior to use of the applicator to apply the fluid 902. In an alternative example, the ampule may include a material (e.g., foam) that is appropriate for patient contact, and the user may use the ampule to apply fluid to the patient.

Regardless of whether the ampule 150 is incorporated with the applicator 12 according to the embodiments of FIG. 47A or 47B, the fluid-containing reservoir 900 is normally in a non-ruptured state in which the fluid 902 is retained in the sealed reservoir. When the applicator is ready for use, the sealed reservoir is ruptured by the user. As noted above, the user may rupture the reservoir 900 by manually squeezing the reservoir with their hand inserted into the cavity 14 or by otherwise applying pressure to the ampule to rupture the reservoir (e.g., by breaking a frangible reservoir). FIGS. 47D and 47E illustrate the ampule 150 in isolation with the fluid-containing reservoir 900 ruptured. When the reservoir 900 ruptures, the fluid 902 immediately flows into the space 912. The size and number of perforations 908 in the perforated layer 904, as well as the viscosity of the fluid 902, defines a mechanism that the user may rely upon to control the amount and rate at which fluid 902 is dispersed from the ampule 150 onto the agent application material 24. The perforations and the viscosity of the fluid may further be useful in controlling excessive release of fluid onto the agent application material 24 upon rupture of the fluid-containing reservoir 900.

With reference to FIGS. 47F and 47G, with the fluid-containing reservoir 900 ruptured and fluid 902 in space 912, the user continues to squeeze the ampule 150 so that the fluid 902 disperses through the perforations 904, thereby soaking the agent application material 24 with the fluid. The user is easily able to control the amount of fluid that is thus dispersed onto the agent application material—squeezing the ampule 150 very tightly causes more fluid 902 to flow out of space 912 through perforations 904 and thus transfer onto the agent application material—arrows A on FIGS. 47F and 47G. As such, the ampule 150 defines a controlled dispersion mechanism whereby the user is able to control the amount of fluid 902 that is deposited onto the agent application material 24 or other target surface.

The user thus selectively controls the amount of fluid that is dispersed from the ruptured ampule 150 onto the agent application material. When additional fluid 902 is required for scrubbing a patient, for example, the user may disperse additional fluid 902 onto the agent application material by continued squeezing of the ampule. During the initial rupture of the fluid-containing reservoirs described herein, in which the fluid may be placed under relatively high pressure, the space 912 provides an area for the liquid to fill when it “bursts” out of the reservoir. Once the fluid is in the space 912 and the pressure on the fluid has been reduced, the user can then control the dispersion of the fluid through one or more perforated layers.

In an example applicator without a perforated layer between the fluid-containing reservoir and the agent application material, when enough pressure is applied to open the reservoir and release the fluid, liquid may be released quickly onto the agent application material. The presence of a perforated layer between the reservoir and the agent application material, however, may help contain the initial flow of fluid, which may prevent an abrupt release of fluid onto the agent application material. The user may then control the rate of fluid flow or transfer from a first side of the perforated layer facing the reservoir to a second side of the perforated layer facing the agent application material. Accordingly, a perforated layer as described in the various examples herein may prevent an initial burst of fluid onto an application surface and may allow the user to control the rate of fluid flow or transfer onto the application surface.

The number, size and position of perforations 908 may be varied to control the rate and direction of dispersion of fluid 902 through the perforated layer 904 and thus onto the agent application material 24. Three different examples of this are illustrated in FIGS. 47H, 47I and 47J. In FIG. 47H, the size of perforations 908 and their locations on layer 904 varies. For example, the perforations 908 near a first end of the reservoir 900 may be relatively smaller, and the perforations 908 may progress in size towards a second end of the reservoir 900. In FIG. 47I, the size and placement of the perforations 908 is similarly varied. For example, the perforations 908 may be concentrated in a specific region of the perforated layer 904, while other areas of the perforated layer 904 do not include perforations 908. Furthermore, within the region that includes perforations 908, the perforations may vary in size. In one example, a relatively larger perforation 908 may be surrounded by a first ring of smaller perforations 908. A second ring of even smaller perforations 908 may lie outward relative to the first ring of perforations 908. And in FIG. 47J, perforations 908 may be formed in only one surface or side of the perforated layer 904. In one example, the perforated layer 904 may include perforations in a surface that faces towards the agent application material 24 to allow the fluid to be directed towards the agent application material 24.

FIG. 47K illustrates a further example of an ampule, including a fluid-containing reservoir 900 and a layer 904a. FIG. 47K diagrammatically illustrates that the perforated layer 904 may include a material having openings in the form of pores (e.g., passageways) that allow fluid to pass through. The pores may remain normally open, or they may be closed and be configured to open upon the application of pressure. The openings through the wall of a layer may therefore include perforations that are cut or formed in a material, as shown in FIG. 47A, or the openings may include pores through a porous material, as shown diagrammatically in FIG. 47K. Accordingly, as noted above, any of the examples described herein that include a perforated layer 904 having openings described as perforations 908 may alternatively include a layer 904a having openings described as pores. In one example, the layers 904 or 904a may include a previously continuous material that becomes permeable when it is heat sealed to the agent application material 24.

Referring to FIG. 47L, layers of material in addition to perforated layer 904 may be used to control fluid flow or transfer from the reservoir 900 to the agent application material 24. FIG. 47L illustrates an applicator 12 similar to the applicator shown in FIG. 47A, with the addition of a layer 914. The layer 914 may lie between the reservoir 900 and the agent application material 24 and may be adjacent the perforated layer 904. Although shown exterior to the perforated layer 904, the layer 914 may lie interior to the perforated layer 904 (e.g., between the reservoir 900 and the perforated layer 904). The layer 914 may include material with openings or seepage qualities, although for clarity they are not shown in FIG. 47L. The openings of layer 914, like the perforations 908 of perforated layer 904 or the openings of layer 904a, may include any type of opening through the material and may be formed in any manner. For example, the openings may be pores of a porous material, or cuts on a previously continuous material.

The openings of layer 914 may be the same or different in size relative to the perforations 908 of perforated layer 904. Furthermore, the layer 914 may have openings in a greater concentration or density than the perforations 908 of perforated layer 904. In one example, the openings of layer 914 are smaller and more concentrated than the perforations 908 of perforated layer 904. The openings of layer 914 may be offset relative to the perforations 908. For example, one or more openings of layer 914 may overlie regions of perforated layer 904 that do not include perforations 908. In this manner, layer 914 may slow the passage of fluid 902 to the agent application material 24 and provide additional dispersion control to the user. Variations (e.g., size, density) between the perforations of perforated layer 904 and the openings of layer 914 may cause fluid to flow through each layer at a different rate.

FIG. 47M illustrates another example of an applicator that may include multiple layers of material to control fluid flow or transfer from the reservoir 900 to the agent application layer 24. In FIG. 47M, a layer 916 may surround the reservoir 900 within the pocket 906. The layer 916 may include openings 918, which may include any of the features of the perforations 908 of perforated layer 904 or the openings of layer 914 described in connection with FIG. 47L. Furthermore, the layer 916 may include any of the materials of perforated layer 904 or layer 914. The layer 916 may enclose all sides of the reservoir 900, completely surrounding reservoir 900. However, in other examples, the layer 916 may be placed exterior to some regions of the reservoir 900 while leaving other regions uncovered by layer 916. This selective placement of layer 916 may slow fluid flow in those regions covered by layer 916 and allow faster fluid flow in the uncovered regions.

Referring to FIG. 47N, a fluid-containing reservoir 900 according to any of the examples described herein may be positioned exterior to the inner liner 22 and interior to an outermost edge of the agent application material 24. For example, the reservoir 900 may be embedded in agent application material 24 or may lie between the inner liner 22 and the agent application material 24.

FIGS. 47O and 47P illustrate applicators 12 according to any example described herein, although the applicators are shown with ampules having a fluid-containing reservoir 900 and perforated layer 904. Enclosures 919, 921 may be enclosures of any shape and may each enclose one or more applicators 12. For example, enclosure 919 may be a container having a generally rectangular shape. Enclosure 921 may be a package similar to the external sheaths described in various examples herein and may include any features described herein in connection with external sheaths. However, the enclosures may include any other shape (e.g., round, oval, irregular). The enclosures 919, 921 may include paper, film, glass, plastic, Tyvek®, or any other suitable material for protecting applicator 12 from contamination. The enclosures 919, 921 may be any height, as illustrated by the taller height of the enclosure 919 relative to enclosure 921, and may accommodate an applicator in an expanded position (FIG. 47O) or in a collapsed position (FIG. 47P).

Referring to FIG. 47O, each enclosure may include a body portion 925 and a top 923. The top 923 may be any cover that prevents contaminants from entering the interior of a enclosure. In one example, the top 923 may include a flexible material, such as paper, plastic, or Tyvek® that may be peeled away from the body portion 925. The top 923 may be selectively permeable to sterilization techniques, such as those using ethylene oxide. The top 923 may be removably sealed by glue, heat, or another material to the body portion 925. In an alternative example, the top 923 may have a lip that fits around the edges of the body portion 925, and the lip may be peeled away from the edges to separate the top 923 from the body portion 925.

FIGS. 47Q-47T illustrate various mechanisms for rupturing a fluid-containing reservoir according to any of the examples described herein, although the reservoirs will be given new reference numbers for ease of description. Referring to FIG. 47Q, the material of reservoir 920 may be flexible and may be configured to rupture upon application of a sufficient force. Accordingly, when the user squeezes his or her hand to draw the ends of the reservoir 920 towards each other, pressure on the outer wall 922 may build sufficiently to cause a region of the wall 922 to rupture, allowing fluid 902 to exit the interior of reservoir 920. To help control the release of fluid, the material of the reservoir 920 may be selected to rupture at a pre-selected pressure.

Referring to FIG. 47R, the material of reservoir 924 may include a weakened region 926. The weakened region 926 may be a seam created during formation of reservoir 924 or may be a line or area of a wall of reservoir 924 having a thickness that is less than a thickness of other walls of reservoir 924. For example, the weakened region 926 may be a laser-etched line extending along all or a portion of the length of reservoir 924, which causes the region of the laser-etched line to be thinner than other walls of the reservoir 924. When the user squeezes the ampule 150, the reservoir 924 may rupture along the weakened region 926, allowing fluid 902 to exit the interior of reservoir 924.

FIGS. 47S and 47T illustrate reservoirs that may include structure to facilitate perforation of the wall of the reservoir. The reservoir 928 of FIG. 47S may include an opener 930 coupled to a body portion 929. The opener 930 may include an elongated portion 934 coupled to the body portion 929 by a protrusion 932. The opener 930 may include a more rigid material than the body portion 929. Accordingly, when a user squeezes the reservoir 928, one or both ends of the elongated portion 934 of the opener 930 puncture the walls of the body portion 929.

Referring to FIG. 47T, a reservoir 936 may include multiple openers 938 coupled to the body portion 929. Each opener 938 may include two legs extending from the wall of the body portion 929 and a central portion coupled to each of the two legs. Similar to opener 930, openers 938 may include a more rigid material than the body portion 929. Accordingly, when a user squeezes the reservoir 928, one or more legs of the openers 938 may puncture the wall of the body portion 929.

The openers shown in FIGS. 47S and 47T may be made of any material suitable to create an opening or rupture in the material of body portion 929. Furthermore, the openers may include any suitable shape or object, including, pins, needles, or edges (sharp or blunt), configured to create an opening or rupture in the body portion when sufficient pressure is applied. The openers may be coupled to the body portion of a reservoir by any suitable means, including by adhesives, heat bonding, or mechanical coupling using a snap, hooks, components that may be slidably coupled, etc. In another example, the opener may be coupled to another component of the applicator 12, such as the inner liner 22 or any other material that is adjacent to the reservoir. In yet another example, the opener may be contained within the fluid-containing reservoir and/or connected to an interior wall of the reservoir.

In yet another example, a fluid-containing reservoir may include a plurality of openings in its walls. The openings may be sized appropriately to retain fluid in the reservoir absent the application of pressure. However, when the user applies pressure to the reservoir, the fluid may flow out of the openings in the walls of the reservoir. Accordingly, the reservoir is configured to release fluid from the reservoir upon the application of pressure to the reservoir.

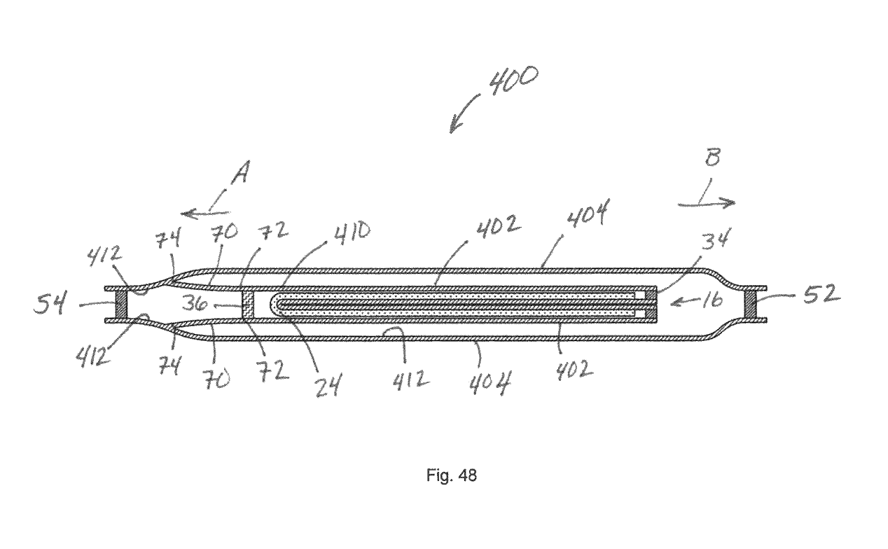

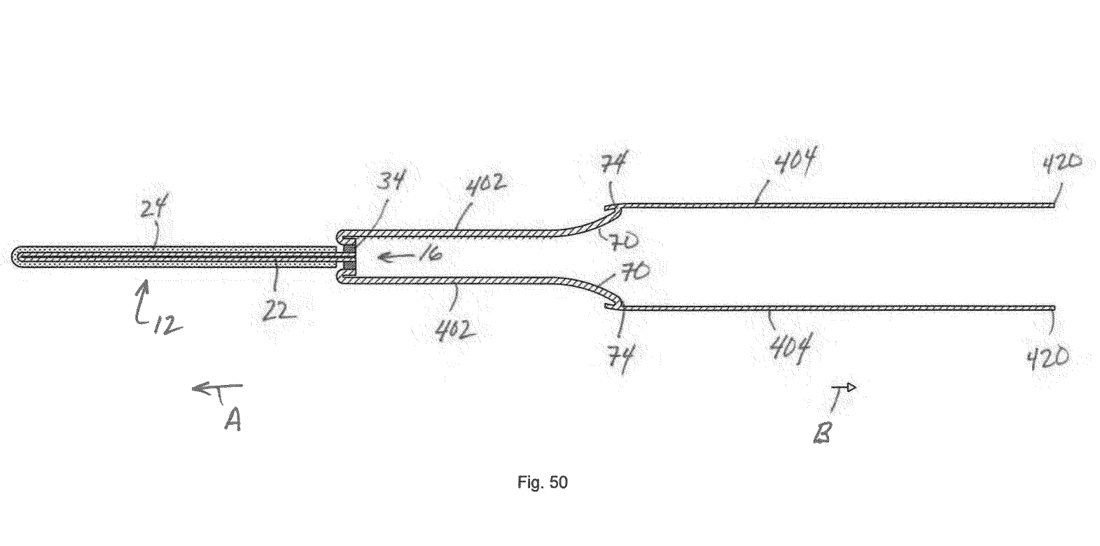

Attention is now drawn to the embodiment of a skin prep delivery system 400 shown in the three cross sectional views of FIGS. 48 through 50. Here, the restrictor strips 72 have been relocated and positioned adjacent end 20 of applicator 12. As detailed below, with this embodiment the external sheath 404 defines an arm-covering sleeve 404 that extends up and over a user's arm when the system 400 is deployed. Specifically, beginning with FIG. 48, each of the two restrictor strips has a first end 72 permanently or semi-permanently attached to the protective sheath 402 that encases applicator 12 adjacent releasable seal 36. As illustrated in the drawing, the strips 70 may be defined by an extension of the protective sheath 402, or alternately, they may be separate strips. The first of the two restrictor strips 70 has its first end 72 attached to the outer surface 410 of the protective sheath 402, or as illustrated, is an extension of the sheath. The second of the two restrictor strips 70 likewise has its first end 72 attached to the outer surface 410 of the protective sheath, but on the other side of the sheath so that releasable seal 36 is located between the two strips 70. The second end 74 of each strip 70 is permanently or semi-permanently attached to the inner surface 412 of external sheath 404. Thus, the second end 74 of the first of the two restrictor strips 70 is attached to inner surface 412 near breakable seal 54, and the second end 74 of the second of the restrictor strips 70 is attached to inner surface 412 opposite breakable seal 54 so that the seal is located between the two strips.

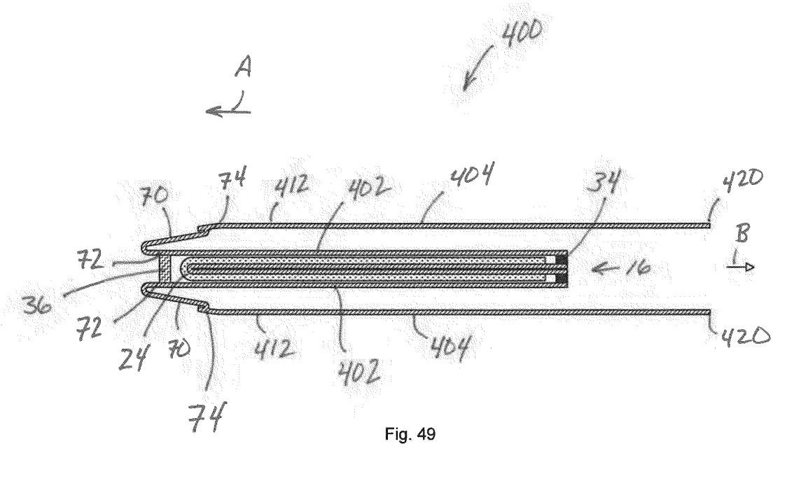

It will be appreciated that to deploy system 400, a user first breaks seal 54, then breaks seal 52 at the opposite, proximal end of the system, and inserts his hand through opening 16 and into cavity 14 of applicator 12. This first stage of deployment of system 400 is shown in FIG. 49 where both seals 52 and 54 have been broken and the external sheath 404 has been pulled toward the user, as shown with arrow B in FIG. 49. This causes the strips 70 to fold back over themselves as shown. In this first stage of deployment the aseptic field within the protective sheath 402 is still intact. The user then grasps the edge of external sheath 404 that is located nearest the user's wrist, identified with reference number 420, and simultaneously pushes his hand forward (arrows A, FIGS. 48 through 50) while pulling external sheath 404 rearwardly (arrows B, i.e., upwardly over the user's arm). As the applicator is translated from right to left in the drawing, seal 36 is broken by the adjacent tip of applicator 12 and the protective sheath 402 is turned inside-out and translated from left to right in the drawings to separate the sheath from the applicator and to define a sleeve as the protective sheath and external are pulled over the arm. Depending upon the size of the system 400, the sleeve 404 can extend significantly up the user's arm, up to a point near the user's arm pit. The action of the user deploying the protective sheath 402 and external sheath 404 so that they cover the user's arm is akin to pulling a sleeve onto an arm. Moreover, once the applicator is fully deployed the user may remove the external sheath 404 if the seal at second end 74 is semi-permanent. An alternative way of deploying applicator 12 with system 400 is to form a strong attachment between protective sheath 402 and external sheath 404 near end 72, but only on one side of the system. With this embodiment, when the user pulls back at edge 420 there will be a tear near end 72, with the same result as described above.

Yet another embodiment of a skin prep delivery system 500 is illustrated in cross sectional view in FIG. 51. In this embodiment the external sheath 504 defines a sterile or non-sterile enclosure for the applicator 12, and the applicator and the protective sheath 502 as detailed below (a) defines an aseptic enclosure for the applicator, and (b) is not attached in any way to the external sheath, either permanently or releasably. In other words, the applicator 12 is free floating within the enclosure defined by the intact external sheath 504. In FIG. 51 the protective sheath 502 is a continuous and contiguous extension of inner liner 22 of applicator 12.

External sheath 504 has a breakable seal 52 at one end thereof. The seal 54 at the opposite end may be either permanent, that is, not breakable, or breakable. The protective sheath 502 is defined by a tubular sheet that is preferably a contiguous extension of the inner liner 22 (although it may be a separate tubular member that is attached to the inner liner 22 around the opening 16 to cavity 14). The tubular sheet 502 extends forwardly, toward the finger portion of the applicator 12 (i.e., to a point 508 that is forward of the distal end 510 of the applicator). At point 508 the inwardly facing surfaces of the tubular protective sheath 502 are sealed together with a breakable, releasable seal 512. The tubular sheath 502 is then folded over and extended rearwardly toward the proximate end 514 of the applicator; the sheath 502 terminates at edge 516. When deployed, the tubular sheath 502 defines an arm covering sleeve.

Antiseptic delivery system 500 is deployed by breaking the seal 52. The user then inserts her hand through opening 16 and into applicator 12. The user then removes external sheath 504—arrow A. At this point with the sheath 504 removed, the user grasps sheath 502 at edge 516 and pulls the sheath over their wrist and arm—arrow B. The distal end 510 of the applicator breaks the seal 512 and the tubular sheath is pulled upwardly over the user's arm with the applicator 12 fully exposed for use. Alternately, the system 500 may be deployed with equal effectiveness by breaking seal 52 and laying the entire device on a flat surface. The user then peels off the upper layer of external sheath 504. The applicator 12 is removed, all the while taking care to maintain the sterility of the area near point 508, for instance, by grasping at edge 516 and avoiding contact with the area near point 508.

In delivery system 500 illustrated in FIG. 51 the tubular sheath 502 is folded over onto itself only once when the system is in the non-deployed state. It will be appreciated that the length of the sheath may be increased, in which case the sheath may require folding over onto itself more than once in the non-deployed condition.

As noted previously, in some circumstances it is not possible to maintain an aseptic environment in the space between the external sheath and the protective sheath. If this occurs, the applicator 12 could be contaminated when the apparatus 10 is deployed from its storage state to its in-use state. In other words, with reference to the embodiment of FIG. 9, if the space between external sheath 46 and protective sheath 30 is not sterile, when the user breaks seal 36 to deploy the applicator 12, the applicator could become contaminated. To eliminate this problem reference is now made to the series of FIGS. 52 through 58 that illustrate one possible structure that prevents contamination of the applicator 12 as it is moved from its non-deployed position to its deployed position when the conditions externally of the protective sheath are not aseptic.

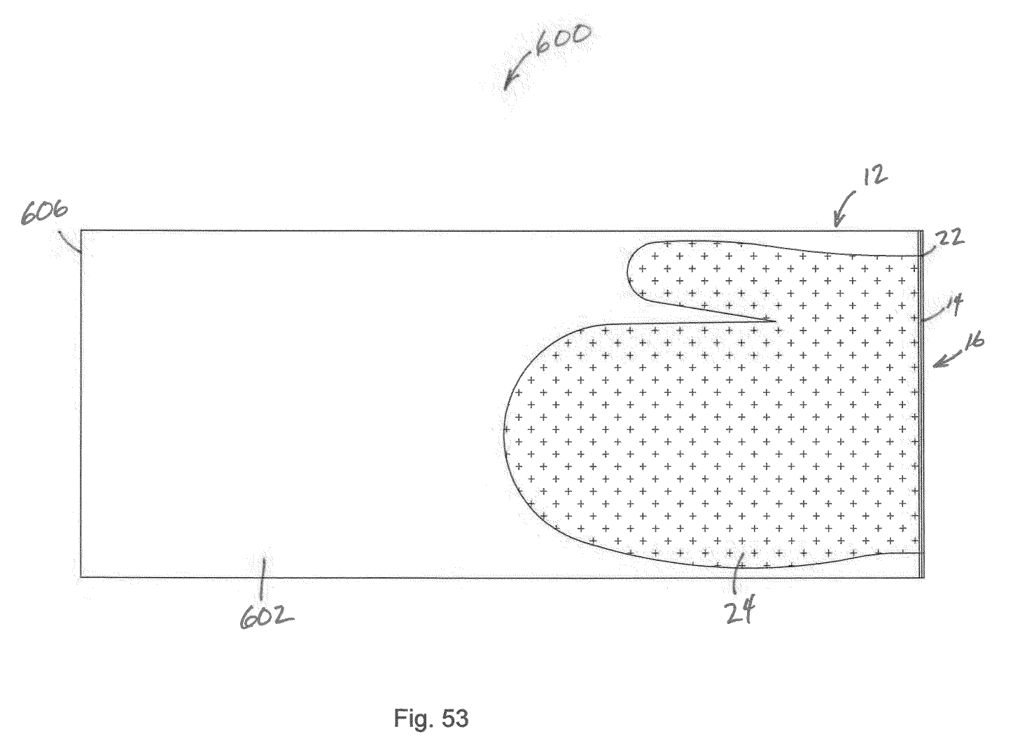

The skin prep delivery system 600 shown in FIG. 52 is analogous to the skin prep delivery system 500 shown in FIG. 51 insofar as the protective sheath 602 is a tubular sheet that is attached to the liner 22 of applicator 12 and the sheath, when deployed as in FIG. 52, defines a sleeve that covers the user's arm. FIGS. 52, 53, 54, 55 and 56 illustrate a sequential series of steps that are followed during assembly of the system 600. In FIG. 53 the protective sheath 602 has been folded over the applicator 12 so that the applicator is contained within the tubular sheet and the opening 16 into cavity 14 at proximal end 604 is exposed so that a user may insert their hand into the applicator. The open end edge of the tubular protective sheath 602 is at the left in FIG. 53 and is labeled with reference number 606.

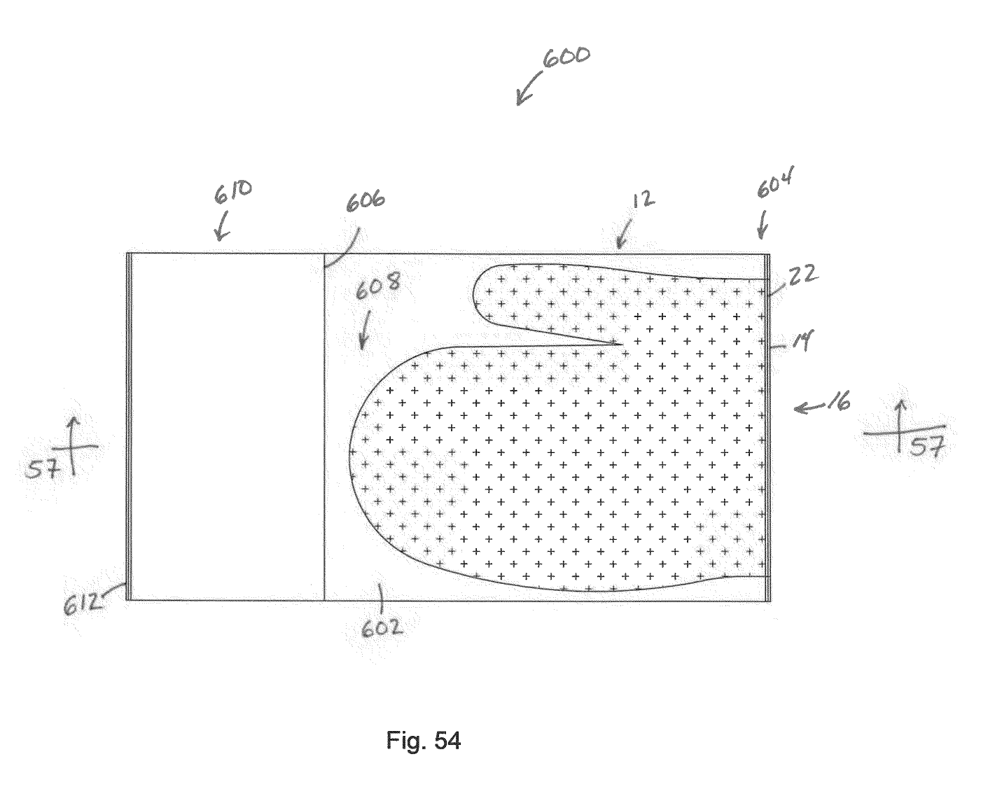

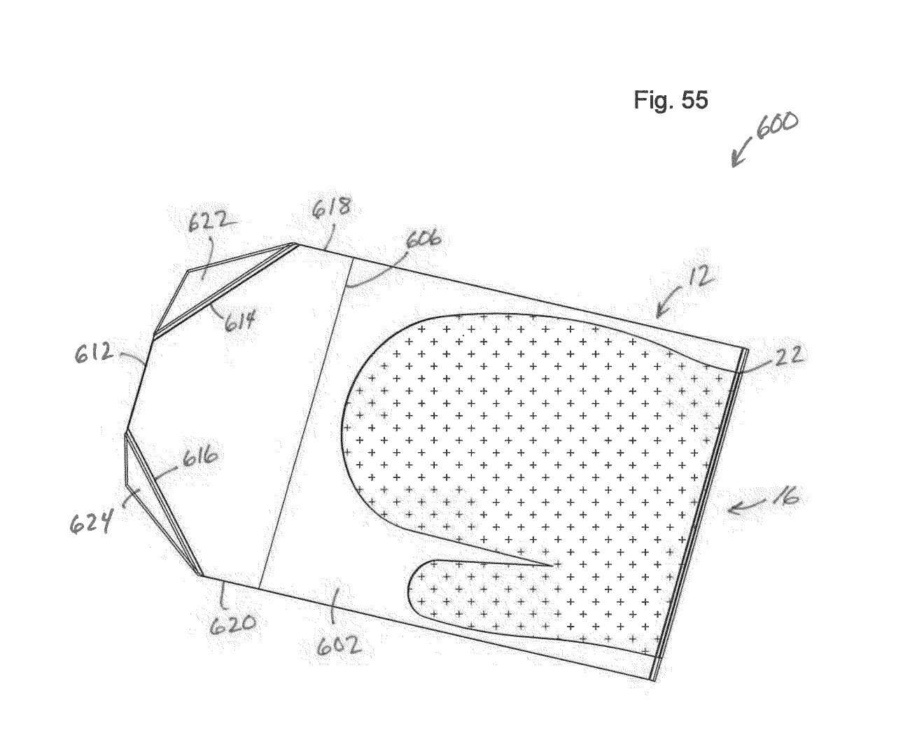

In the next sequential drawing, FIG. 54, the open end edge 606 of the protective sheath 602 has been folded back over itself, in the direction toward the applicator 12 with the end edge 606 located adjacent the distal end 608 of applicator 12. This creates a double-layered region 610 between end edge 606 and the new folded over end 612. Moving to FIG. 55, two folds are formed along diagonal fold lines 614 and 616. Specifically, fold line 614 extends diagonally from folded over end 612 to side edge 618 in the double-layered region 610, and fold line 616 extend diagonally from folded over end 612 to opposite side edge 620, also in the double-layered region 610. The diagonal fold lines create triangular corner sections 622 and 624. The triangular corner sections 622 and 624 are folded downwardly into the plane of the drawing sheet.

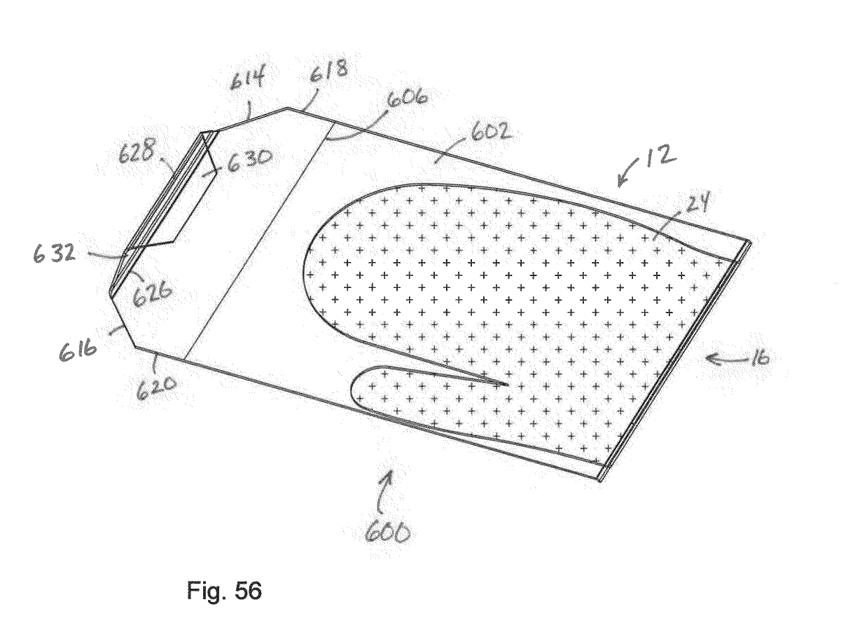

Next, as seen in FIG. 56, the folded over end 612 is folded at two fold lines 626 and 628 between the diagonal fold lines 614 and 616 with the thus formed end flap 630 being tucked under the adjacent flap 632. It will be appreciated that there are numerous variations in the specific folding patterns that will have the same functional result. For example, a fold extending straight across the protective sheath, coupled with a rolled end that uses no diagonal folds will be effective.

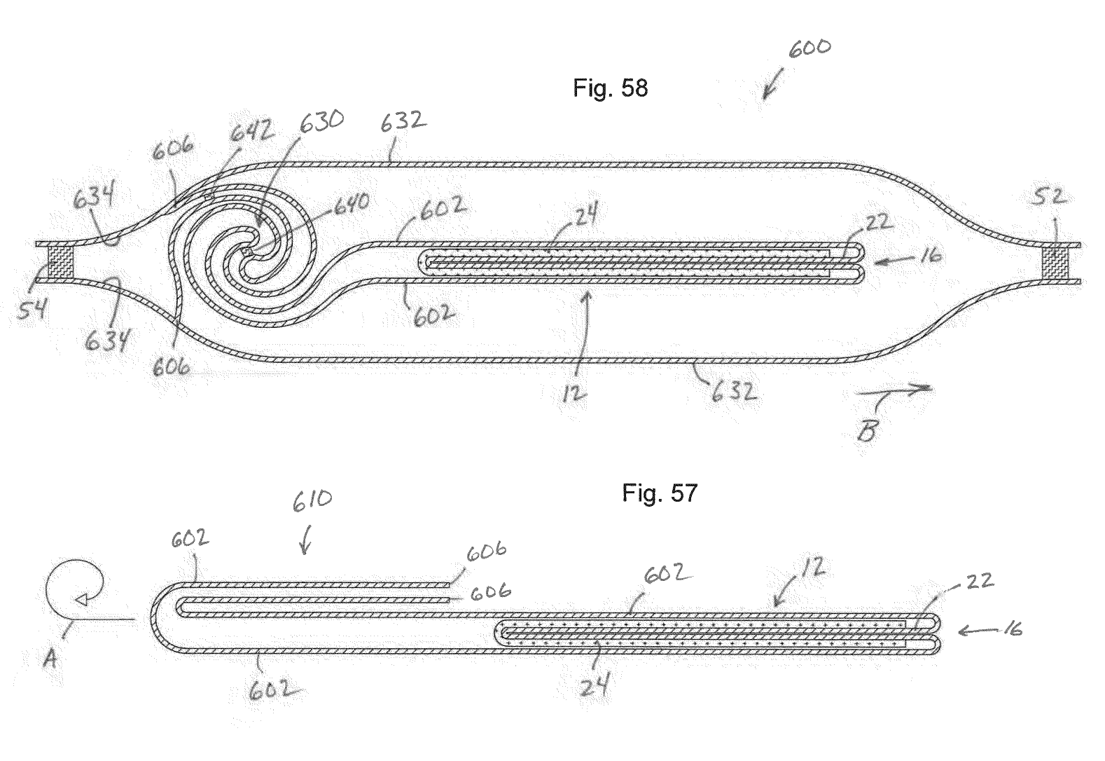

FIG. 57 is a cross sectional and schematic illustration that illustrates the process of rolling up of the end of the tubular protective sheath 602 to define a sterile tip through which applicator 12 is extended from the non-deployed to the deployed position. The circular arrow A is a schematic representation that demonstrates that the 4 layers of sheath 602 are rolled over onto themselves beginning at the distal end 630 and rolling from the four layers of double-layered region 610 from the left to the right in FIG. 57; the rolling is continued for approximately one full turn of the distal end. The applicator 12 and protective sheath 602 with the rolled end shown in FIG. 57 is enclosed in an external sheath 632 as illustrated in FIG. 58. Intermediate seals 640 and 642 are formed between the facing surfaces of the tubular protective sheath 602 in the rolled portion of the sheath. It will be understood that the location of the intermediate seals 640, 642 may be anywhere in the tubular region and that the locations illustrated are exemplary only. The end edges 606 of the tubular sheath are permanently or semi-permanently attached to the interior surface 634 of external sheath 632 as shown, adjacent and on opposite sides of seal 54. The opposite end of external sheath 632 is sealed as discussed above with a seal 52.

With returning reference to FIG. 57, it will be appreciated that the two end edges 606 may be “sealed” to one another with a mechanical attachment such as a clip. The clipped layers may then be rolled.

Use of the system 600 will be evident from the foregoing discussion of other embodiments and the drawings. Briefly described, the seals 54 and 52 are broken and the user inserts her hand into applicator 12 through opening 16. The user pulls the external sheath upwardly over their arm (arrow B, FIG. 58). This causes the distal end 608 of applicator 12 to translate through the tubular protective sheath 602, breaking seals 640 and 642 as it moves through the sheath. As the user continues to pull the sleeve defined by external sheath 632 up their arm, the ends 606 of the sheath begin to be pulled toward the user and the protective sheath 602 thus is turned inside out and forms a part of the sleeve covering the users arm when the applicator 12 is exposed in its deployed position.

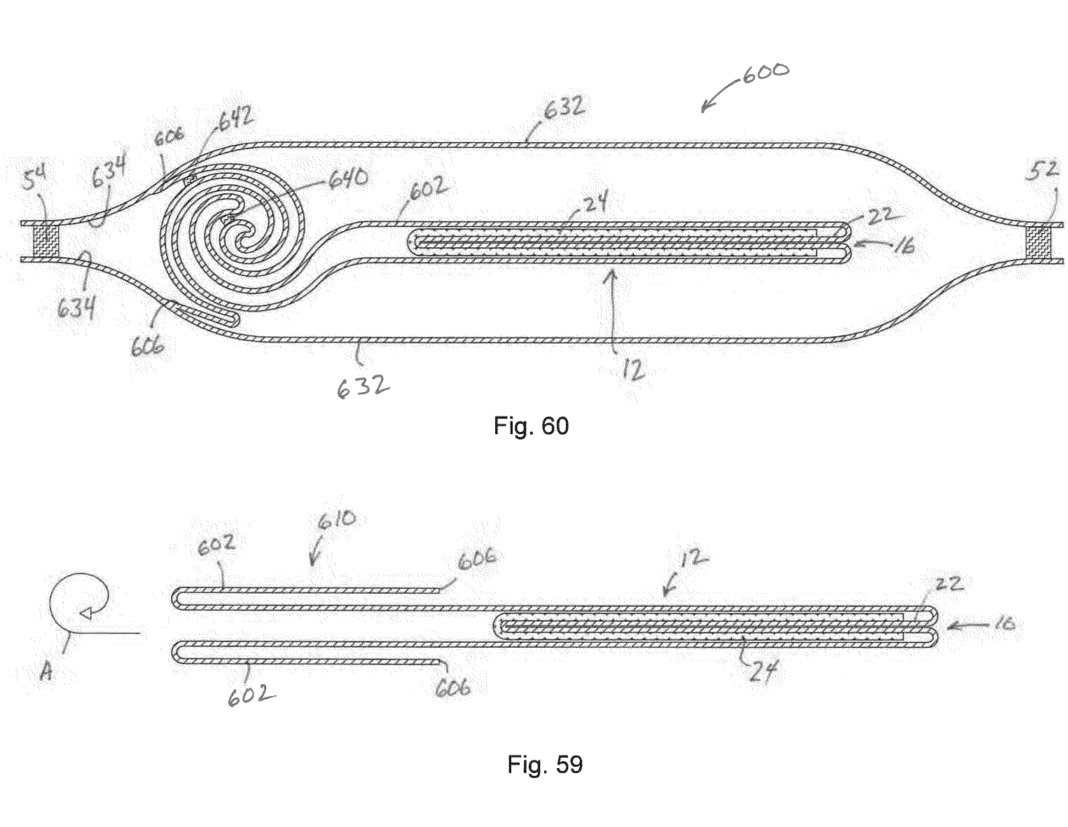

FIGS. 59 and 60 are analogous to FIGS. 57 and 58 but show a slightly modified arrangement that serves the same function, to provide a sterile passageway for the applicator 12 to move through when it is deployed. The difference between FIGS. 57 and 59 is evident: in FIG. 59 the tubular protective sheath 602 is folded back over itself tubularly rather than folded over as shown in FIG. 57. The embodiment of FIGS. 59 and 60 is otherwise the same and the end edges 606 are likewise attached to the interior surfaces 634 of external sheath 632. Deployment of the embodiment of FIGS. 59 and 60 from the stored position to the use position is identical to that described above in respect of FIGS. 57 and 58.

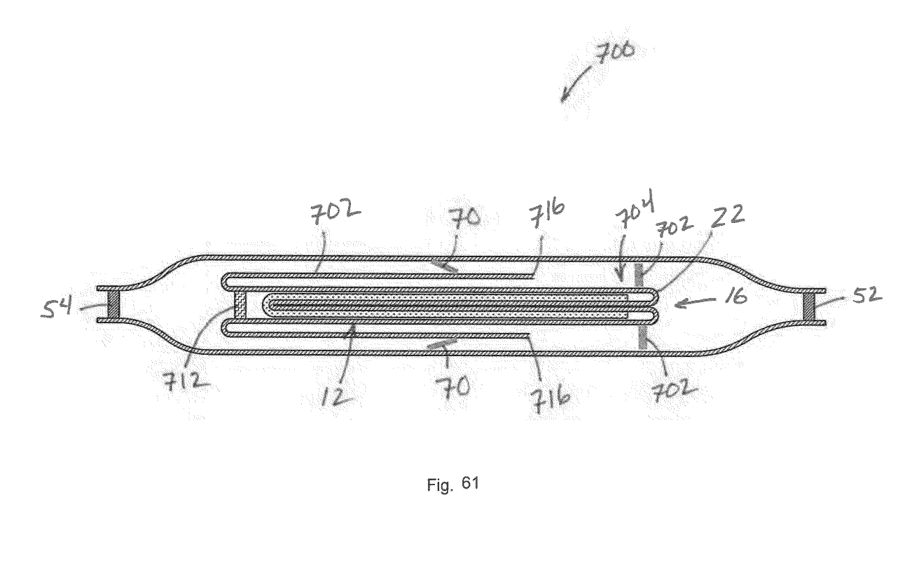

The embodiment of delivery system 700 shown in FIG. 61 is analogous to the skin prep delivery system 500 shown in FIG. 51 insofar as the protective sheath 702 is a tubular sheet that is attached to the liner 22 of applicator 12 and the sheath, when deployed as in FIG. 52, defines a sleeve that covers the user's arm. Delivery system 700 includes the same basic structures as described with respect to system 500, including a releasable seal 712 near the distal end of applicator 12. However, the length of the protective sheath, which terminates at edge 716 is relatively shorter than the analogous length of the sheath in FIG. 51, which terminates at edge 516, and a pair of restrictor strips 70 interconnect the outer surface of the protective sheath to the facing, inner surface of the external sheath. Further, a light and releasable adhesive 702 has been added between the outer surfaces of the protective sheath 702 at the proximate end 704 adjacent the opening 16 and the facing portion of the external sheath. The adhesive 702 insures that the user's hand is directed into the opening 16 into applicator 12, but readily releases when the system is being used.

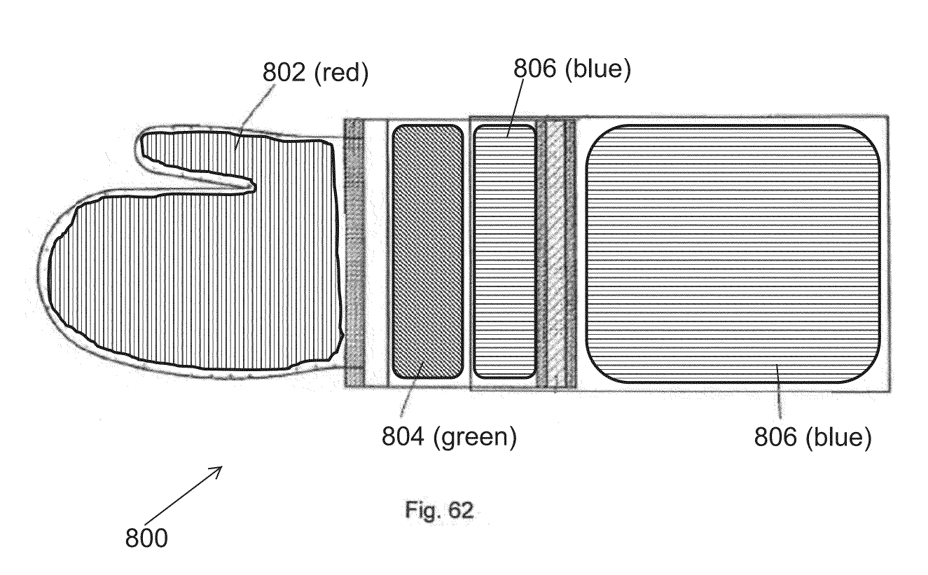

In some circumstances the delivery system will be simpler to use when a colour coding system is utilized—the colours immediately indicate to a user which parts of the system are aseptic (and therefore should not be touched), which are clean (but not necessarily medically sterile) and which are not medically clean (i.e., appropriate for handling by the user). With reference to FIG. 62, a delivery system 800 is illustrated with cross hatching to schematically illustrate the colours that may be applied to the device to immediately convey to the user the relative sterility and cleanliness of the device. Most importantly, the applicator portion of the system 800, that is, the mitt 802 that is used to scrub the patient, is colour coded in RED. The protective sheath portion, which in the view of FIG. 62 is labeled with reference number 804 and is colour coded GREEN, a colour which is intended to indicated to the user that the portion that is green is medically clean but is not medically aseptic. Immediately to the right of protective sheath 804 is external sheath 806, which is colour coded BLUE. This colour indicates to the user that it may be handled and manipulated by the user during use without concern about compromising sterility. The system 800 in FIG. 62 is of course illustrated in its deployed position. It will be recognized and appreciated that the colour coding system just described and shown in FIG. 62 carries over to the system 800 when it is in its storage, non-deployed condition. Stated another way, the various portions of the system that are coloured may be seen when the system is in the non-deployed condition, and the user will thus recognize the relative sterility and cleanliness conditions of the device. The relative colours that are used for the various sections of the system are not important—the mitt 802 could be GREEN and the external sheath 806 could be RED. The important aspect is the colour differential that the user recognizes as having meaning with respect to the relative cleanliness of the device.

### USES

Although the present disclosure describes a disinfecting system for use in antiseptic, it is contemplated that the disinfecting system can be used for cleaning or disinfecting or applying a fluid or agent to any surface of interest. The system can be adapted for use as a general surface cleaner for medical, pharmaceutical, industrial or household applications, for example, by pre-loading the applicator 12 with an appropriate fluid or agent for the particular application of interest. It will be readily understood by persons skilled in the art that such applications may not necessarily require that the system maintain aseptic conditions prior to use, or require pre-loaded fluid or agent.

It is contemplated that any embodiment discussed herein and shown in the various drawings may be implemented with respect to any method or composition of the disclosed examples. Furthermore, compositions and kits of the disclosed examples can be used to achieve exemplary methods of the disclosure.

The disclosures of all patents, patent applications, publications and database entries referenced in this specification are hereby specifically incorporated by reference in their entirety to the same extent as if each such individual patent, patent application, publication and database entry were specifically and individually indicated to be incorporated by reference.

Although the aspects of the disclosure have been described with reference to certain specific examples, various modifications thereof will be apparent to those skilled in the art without departing from the spirit and scope of the disclosure. All such modifications as would be apparent to one skilled in the art are intended to be included within the scope of the following claims.

We claim:

1. An agent delivery system, comprising:
an applicator including a hand piece having a hand-receiving cavity and an agent application material on a surface of the hand piece; and
a fluid-containing reservoir, wherein the fluid-containing reservoir is configured to release fluid from the reservoir to allow the fluid to transfer onto the agent application material.

2. The agent delivery system of claim 1, further comprising a layer having a plurality of openings, wherein the layer is positioned between the fluid-containing reservoir and the agent application material.

3. The agent delivery system of claim 2, wherein the plurality of openings includes at least one of perforations or pores.

4. The agent delivery system of claim 2, further comprising a fluid-collecting volume between the fluid-containing reservoir and the layer.

5. The agent delivery system of claim 2, wherein the layer is a first layer, and the system further comprises a second layer having a plurality of openings, wherein the second layer is positioned between the fluid-containing reservoir and the agent application material.

6. The agent delivery system of claim 2, wherein the layer encapsulates the fluid-containing reservoir.

7. The agent delivery system of claim 1, wherein the hand piece further comprises a fluid-impermeable inner liner, and the fluid-containing reservoir is between the fluid-impermeable inner liner and the agent application material.

8. The agent delivery system of claim 1, further comprising an enclosure encasing the applicator when the applicator is in a non-deployed position.

9. The agent delivery system of claim 8, wherein the enclosure includes a sheath having at least one seal adjacent to at least one end of the sheath.

10. The agent delivery system of claim 8, wherein the enclosure includes a container having a body portion and a removable top.

11. The agent delivery system of claim 1, further comprising an opener configured to rupture a wall of the fluid-containing reservoir.

12. The agent delivery system of claim 11, wherein the opener is configured to rupture a wall of the fluid-containing reservoir when a first end of the reservoir and a second end of the reservoir are brought towards each other or when the reservoir is deformed by an application of pressure.

13. The agent delivery system of claim 1, wherein the fluid-containing reservoir includes a weakened region configured to rupture.

14. An agent delivery system, comprising:
an applicator including a hand-receiving cavity having an inner lining;
an enclosure encasing the applicator when the applicator is in a non-deployed position; and
a fluid-containing reservoir positioned interior to the enclosure.

15. The agent delivery system of claim 14, wherein the fluid-containing reservoir is positioned interior to an outer surface of the applicator, and further comprising at least one layer having a plurality of openings, wherein the at least one layer is positioned between the fluid-containing reservoir and the outer surface of the applicator.

16. The agent delivery system of claim 15, wherein the at least one layer having a plurality of openings includes a first layer and a second layer, and fluid is configured to flow through the first layer at a different rate than fluid is configured to flow through the second layer.

17. The agent delivery system of claim 15, wherein the at least one layer is a portion of the inner lining.

18. An agent delivery system, comprising:
a hand piece having a hand-receiving cavity;
an agent application material on a surface of the hand piece;
a compartment, wherein a portion of the wall of the compartment includes a layer having a plurality of openings; and
a fluid-containing reservoir positioned in the compartment,
wherein the fluid-containing reservoir is configured to release fluid from the reservoir to allow the fluid to transfer through the layer and onto the agent application material.

19. The agent delivery system of claim 18, wherein the layer is a first layer, and the system further comprises a second layer having a plurality of openings, and the second layer is positioned adjacent to the first layer.

20. The agent delivery system of claim 18, wherein the layer is a first layer, and the system further comprises a second layer having a plurality of openings, and the second layer completely surrounds the fluid-containing reservoir.
